(19) United States
(12) Patent Application Publication
(10) Pub. No.: US 2018/0291123 A1
(43) Pub. Date: Oct. 11, 2018

(54) DI-FUNCTIONALIZED HYALURONIC ACID DERIVATIVES AND HYDROGELS THEREOF

(71) Applicant: THE BROAD OF REGENTS OF THE UNIVERSITY OF TEXAS SYSTEM, Austin, TX (US)

(72) Inventors: Laura A. SMITH CALLAHAN, Houston, TX (US); Hyun Ju LIM, Houston, TX (US)

(21) Appl. No.: 15/757,256

(22) PCT Filed: Sep. 2, 2016
(86) PCT No.: PCT/US2016/050074

§ 371 Date: Mar. 2, 2018

Related U.S. Application Data
(60) Provisional application No. 62/214,598, filed on Sep. 4, 2015.
(60) Provisional application No. 62/291,970, filed on Feb. 5, 2016.

(51) Int. Cl. C08B 37/08; A61K 31/728; A61K 9/06; A61K 47/64; A61K 51/06; A61K 49/00; A61K 35/545; A61K 35/30; C12N 5/0793; C12N 5/079; A61L 27/52; A61L 27/54; A61L 27/38; A61L 27/36; A61L 27/20; A61L 27/50; A61L 27/58; A61P 25/00

(52) U.S. Cl. CPC C08B 37/0072; A61L 2430/38; A61K 9/06; A61K 47/64; A61K 51/065; A61K 49/0054; A61K 35/545; A61K 35/30; C12N 5/0619; C12N 5/0622; A61L 27/52; A61L 27/54; A61L 27/3895; A61L 27/3878; A61L 27/383; A61L 27/3675; A61L 27/3633; A61L 27/20; A61L 27/50; A61L 27/58; A61P 25/00; C12N 2506/08; A61L 2430/32; A61K 31/728

(57) ABSTRACT

Provided herein are di-functionalized hyaluronic acids, such as molecules including (or that have been functionalized at) a thiol and azide side chain. Also, provided herein are hydrogels of these di-functionalized hyaluronic acids and methods of using these compounds to promote cell (e.g., neuronal cell) growth and development. In some aspects, the present disclosure also provides methods of treating injuries, including brain injuries such as stroke through the use of hydrogels of the compounds described herein and stem cells.

This application claims the benefit of U.S. Provisional Application Ser. No. 62/214,598 filed on Sep. 4, 2015 and 62/291,970 filed on Feb. 5, 2016.

### INCORPORATION OF SEQUENCE LISTING

The sequence listing that is contained in the file named “UTFHP0313WO_ST25.txt”, which is 4 KB (as measured in Microsoft Windows®) and was created on Sep. 1, 2016, is filed herewith by electronic submission and is incorporated by reference herein.

### BACKGROUND OF THE INVENTION

### 1. Field of the Invention

The present invention relates generally to the field of modified polysaccharides and hydrogels. More particularly, it concerns di-functionalized hyaluronic acid and hydrogels thereof.

### 2. Description of Related Art

Little progress has been made in the treatment and restoration of neurological function in stroke victims. While stem cell therapy has shown potential as a treatment option for neurological damage from strokes and other brain injuries, the development and growth of neuronal stem cells in the lesion has been subpar due to the lack of focus on the relevance of the extracellular matrix in neuronal development. Artificial extracellular matrix (aECM) are potentially useful tools which could address the current issues with stem cell therapies but have not been extensively studied and developed. Therefore, a need still exists to develop an artificial extracellular matrix which can be used to enhance neuronal development.

### SUMMARY OF THE INVENTION

In some aspects, the present disclosure provides di-functionalized hyaluronic acid which contains two different groups which can be used to cross-link and/or polymerize the hyaluronic acid. For example, in some embodiments a di-functionalized hyaluronic acid is provided comprising thiol and azide functionalization (or linking group).

Thus, in some aspects, the present disclosure provides compounds of the formula:

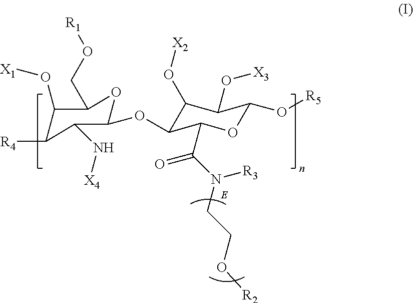

wherein:
R₁ is —Y₁-L₁, wherein:
   Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₁ is a first linking group;
R₂ is —Y₂-L₂, wherein:
   Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₂ is a second linking group;
R₃ is hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
X₁, X₂, and X₃ are each independently hydrogen, alkyl_{(C≤6)}, substituted alkyl_{(C≤6)}, or a hydroxy protecting group;
X₄ is hydrogen, acyl_{(C≤6)}, substituted acyl_{(C≤6)}, or a monovalent amino protecting group;
m is 1-20; and
n is 1-5000;
   provided that the first linking group and the second linking group are different;
   or a pharmaceutically acceptable salt thereof.

In some embodiments, a compound is further defined as:

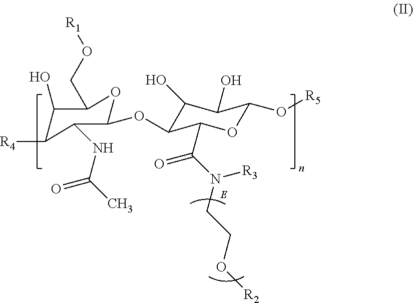

wherein:
R₁ is —Y₁-L₁, wherein:
   Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₁ is a first linking group;
R₂ is —Y₂-L₂, wherein:
   Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₂ is a second linking group;
R₃ is hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
m is 1-20; and
n is 1-5000;
   provided that the first linking group and the second linking group are different;
   or a pharmaceutically acceptable salt thereof.

In further embodiments, the compound is further defined as:

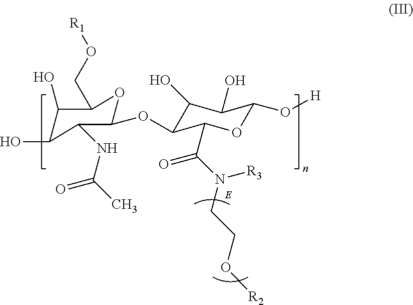

wherein:
R₁ is —Y₁-L₁, wherein:
   Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₁ is a first linking group;
R₂ is —Y₂-L₂, wherein:
   Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₂ is a second linking group;
R₃ is hydrogen, alkyl_{(C≤6)}, or substituted alkyl_{(C≤6)};
m is 1-20; and
n is 1-5000;
   provided that the first linking group and the second linking group are different;

or a pharmaceutically acceptable salt thereof.

In some embodiments, Y₁ is alkanediyl_{(C≤12)} or substituted alkanediyl_{(C≤12)}. In some embodiments, Y₁ is —CH₂CH₂—. In some embodiments, L₁ is —SH. In other embodiments, L₁ is an acrylate or a methylacrylate group. In some embodiments, L₁ is:

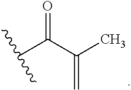 [SMILES: *C(=O)C(=C)C]

In some embodiments, Y₂ is alkanediyl_{(C≤12)} or substituted alkanediyl_{(C≤12)}. In some embodiments, Y₂ is —CH₂CH₂—. In some embodiments, L₂ is —N₃. In some embodiments, R₃ is hydrogen. In some embodiments, m is 2, 3, or 4. In some embodiments, n is an integer between 1 and 1000. In some embodiments, n is an integer between 1 and 500. In some embodiments, the compound is further defined as:

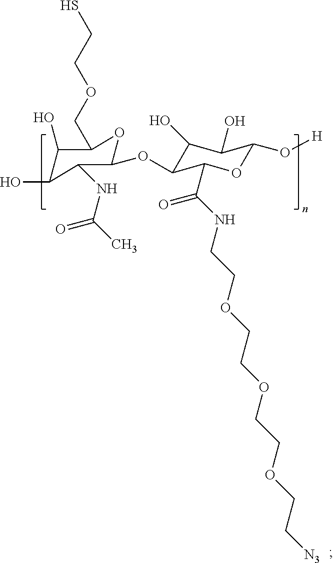

or a pharmaceutically acceptable salt thereof.

In yet another aspect, the present disclosure provides hydrogels comprising a compound described herein. In some embodiments, comprising two hyaluronic units comprising:

a first hyaluronic unit of the formula:

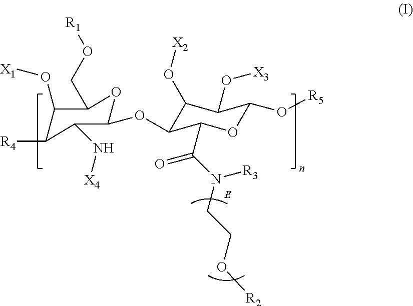

wherein:
R₁ is —Y₁-L₁, wherein:
   Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₁ is a first linking group;
R₂ is —Y₂-L₂, wherein:
   Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₂ is a second linking group;
R₃ is hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
X₁, X₂, and X₃ are each independently hydrogen, alkyl_{(C≤6)}, substituted alkyl_{(C≤6)}, or a hydroxy protecting group;
X₄ is hydrogen, acyl_{(C≤6)}, substituted acyl_{(C≤6)}, or a monovalent amino protecting group;
m is 1-20; and
n is 1-5000; and
   a second hyaluronic unit of the formula:

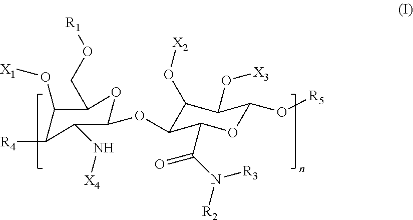

wherein:
R₁ is —Y₃-L₃, wherein:
   Y₃ is a covalent bond, alkanediyl_{(C≤12)}, alkenediyl_{(C≤12)}, alkynediyl_{(C≤12)}, arenediyl_{(C≤12)}, or a substituted version of any of these groups; and
   L₃ is a third linking group;
R₂ and R₃ are each independently hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
X₁, X₂, and X₃ are each independently hydrogen, alkyl_{(C≤6)}, substituted alkyl(C<6), or a hydroxy protecting group;
X₄ is hydrogen, acyl_{(C≤6)}, substituted acyl_{(C≤6)}, or a monovalent amino protecting group;
m is 1-20; and
n is 1-5000;
   provided that the first, second, and third linking groups are all different; and
   wherein the hydrogel comprises a ratio of the first hyaluronic acid to the second hyaluronic acid from about 10:1 to about 1:10.

In some embodiments, the first linking group comprises a thiol unit. In some embodiments, the second linking group comprises an azide group. In some embodiments, the third linking group is a methacrylate. In some embodiments, the ratio is from about 1:1 to about 1:7.5. In some embodiments, the ratio is from about 1:2 to about 1:5.

In some embodiments, the first linking group, the second linking group, or the third linking group is linked to a polypeptide (e.g., an extracellular matrix polypeptide or a growth or differentiation factor). In some embodiments, the polypeptide is derived from laminin, neuroligin-1, fibronectin, collagen, n-cadherin, cartilage oligomeric protein, neural cell adhesion molecule 1, victronectin, brain-derived neurotrophic factor, neurexin, insulin-like growth factor, link binding protein, bone morphgenic proteins, decoriin, or aggrecan binding peptides. In other embodiments, the first linking group, the second linking group, or the third linking group is linked to an imaging agent. In some embodiments, the imaging agent is a chelating group bound to a radioisotope. In other embodiments, the imaging agent is fluorophore. Alternatively, in some embodiments, the polypeptide can be derived from a factor that is known to have activity that has been established to be involved in the intended application, for example when intended for use in orthopedic applications, the polypeptide can be derived from, among others, fibronectin, collagen, n-cadherin, cartilage oligomeric protein, victronectin, insulin-like growth factor, link binding protein, bone morphgenic proteins, decorin, or aggrecan.

In a further aspect, there is provided method of preparing a difunctional hyaluronic acid compound of according to the embodiments comprising: (a) reacting hyaluronic acid with a thiol compound to obtain a thiolated hyaluronic acid derivative; and (b) reacting the thiolated hyaluronic acid derivative with azide containing amine in the presence of a coupling agent to obtain a difunctional hyaluronic acid. In some embodiments the reaction occurs without substantive purification between step (a) and step (b) (e.g., without purification sufficient to isolate the product of the reaction in step (a) before the reaction of step (b)). In still further embodiments, steps (a) and (b) of a reaction are performed in the same reaction vessel. In certain embodiments the thiol compound is ethylene sulfide. In further embodiments the azide containing amine is an azide linked to an amine by a poly(ethylene) glycol repeating unit. For example, the azide containing amine can be 11-azido-3,6,9-trioxaundecan-1-amine.

In still yet another aspect, the present disclosure provides compositions comprising: (a) a hydrogel described herein; and (b) a cell, such as a stem cell. In some embodiments, the cell is a stem cell such as a human induced pluripotent stem (iPS) cell. In further embodiments, the stem cell is a neural stem cell. In some embodiments, the stem cell is encapsulated in the hydrogel.

In another aspect, the present disclosure provides methods of treating a disease or disorder in a patient comprising implant a hydrogel or composition described herein into tissue, such as a diseased or damaged tissue. In certain embodiments the disease or disorder is a neurological disease or disorder. In some embodiments, the neurological disease or disorder is a brain injury. In some embodiments, the neurological disease or disorder is a stroke. In some embodiments, the neurological disease or disorder results in a neuronal lesion. In some embodiments, the methods comprise implanting the hydrogel or the composition described herein into a neuron or neuronal tissue.

In yet another aspect, the present disclosure provides methods of promoting cell or tissue growth by implanting a hydrogel or composition described herein into a tissue under conditions that support cell or tissue growth. In certain embodiments a method is provided for promoting neuronal growth in a neuronal lesion comprising implanting a hydrogel or composition described herein into the neuronal lesion under conditions sufficient to promote neuronal formation. In some embodiments, the methods comprise implanting a composition.

In still yet another aspect, the present disclosure provides methods of inducing differentiation in a cell, such as a neuronal stem cell, comprising contacting the stem cell with a hydrogel described herein under conditions sufficient to cause the stem cell to differentiate into a mature cell (e.g., a mature neuronal cell). In some embodiments, the methods comprise encapsulating the stem cell with the hydrogel. In some embodiments, the mature cell is a mature neuronal cell, such as a neuron, oligodendrocyte, or astrocyte. In some embodiments, the methods are performed in vitro. In other embodiments, the methods are performed in vivo. In some embodiments, the methods are performed in a patient. In some embodiments, the patient is a mammal. In some embodiments, the patient is a human. In some embodiments, the methods comprise implanting the hydrogel or the composition described herein into a neuron or neuronal tissue.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Exemplary schematic of brain ECM for native, injured and artificial extracellular matrix (aECM) supported injured. In the injured ECM proteoglycans seal off the injured area, while enzymes degrade the existing native ECM. This inhibits the migration of cells into and out of the area and leaves implanted cells without a supportive ECM to attach. However, matrix support at the injury site reduces proteoglycan deposition improving cellular migration, survival, and axon extension. The inclusion of a radioactive label allows for visualization in order to mitigate additional injury and allow for implant tracking during recovery

FIG. 2—β3 tubulin staining of mouse embryonic stem cells after 3 days of differentiation on a tethered bioactive laminin peptide (IKVAV; SEQ ID NO: 1) concentration gradient in PEG hydrogels demonstrates a concentration dependent effect on cellular survival, neurite extension and differentiation with toxicity evident at high concentration and lack of neurite extension at low concentration.

FIG. 3—Schematic showing cascade of neural signaling.

FIGS. 4A-C—Three pump cell encapsulation system. A) Schematic of system. B) Picture of pumping system in same orientation as schematic C) Food color gradient generated in 8,000 MW PEGDM hydrogel for demonstration.

FIG. 5—Schematic of di-functional HA modification.

FIG. 6—Schematic of methacrylate HA modification.

FIGS. 7A-B—In reference to the di-functionalized hyaluronic acid (HA) shown in FIG. 5, (B) shows the colormetric quantification of thiol content after functionalization with thiol (HA-SH) and with thiol and azide (HA-SH-AA). (C) is a FTIR of azide peak before and after functionalization.

FIG. 8—Quantification of IKVAV (SEQ ID NO: 1) peptide concentration gradient coupled in PEG hydrogel.

FIG. 9—Autoradiography with a ⁶⁴Cu-radiotracer. Nude mice received U87 brain tumor implants and, after injection of a ⁶⁴Cu-radiotracer, brain tissue was removed, cut into 30 μm sections, and used for autoradiography. Imaging revealed differential tracer uptake within the tumor which co-localized with the site of the tumor implant (indicated by arrows). differential tracer uptake within the tumor (indicated by arrow).

FIGS. 10A-B—Characterization of A) Young's Modulus and B) Material properties of a polyethylene glycol hydrogel containing a continuous gradient in Young's Modulus.

FIGS. 11A-C—Neural differentiation of mouse embryonic stem cells on IKVAV (SEQ ID NO: 1) gradient hydrogel. A) TUJ1 (red) with nuclear staining (blue) after 3 days of neural differentiation. B) Quantification of neurite length after 3 days of differentiation. C) Semi-quantitative PCR of mature neuron maker (MAP2).

FIG. 12—Live (green)/Dead (red) staining of hiPSC derived NSC in 3D culture in PEG hydrogel containing 235 μM IKVAV from gradient after 8 days of neural differentiation.

FIGS. 13A-B—Characterization of thiol group functional HA backbone. (A) 2,4,6-trinitrobenzene sulfonic acid (TBNS) colorimetric assay, and (B) ¹H-NMR for thiol detection on HA, HA-SH, and HA-SH-AA. The 2.8 ppm peak on the ¹H-NMR spectrum and positive response to the TNBS assay were only present on HA samples functionalized with thiol, or thiol and azide groups. (n=3, p*<0.05).

FIGS. 14A-D—Characterization of azide group on HA. (A) FT-IR, (B) isotopic dilution experiment with ⁶⁸Ga, and (C) ¹³C-NMR for thiol detection on HA, HA-SH, and HA-SH-AA. The azide group peak was presented at 2096 cm⁻¹ on the FT-IR spectrum, and at 50.1 ppm on the ¹³C-NMR spectrum. (D) The average number of chelating moieties capping the azide group in HA was 3.9±0.09 on radio TLC on diHA samples.

FIGS. 15A-C—Characterization of diHA/mHA hydrogels of various concentrations. (A) Gelling with 0.1% Irgacure 2959 photoinitiator, (B) Hydrogel properties; Young's modulus, Shear modulus, swelling ratio, and water content on 1:2 and 1:5, diHA:mHA, (C) β-tubulin III positive staining for neuro-differentiation of mES in 1:2 mixing ratio HA hydrogel at Day 3. A higher ratio of mHA increased the Young's modulus and shear modulus. Neural differentiation of mouse embryo stem cells (mES) were examined in 3D HA hydrogels. 44.51±0.4% of mES expressed β-tubulin III on day 3 of differentiation in 1:2 mixing ratio HA hydrogel. (n=5, p*<0.05, p***<0.001).

FIGS. 16A-F—Characterization of diHA hydrogels synthesized using different molecular weight HA. 50,000 mw HA (PEGWORK) versus 60-90,000 mw HA (average molecular weight of batch was 74,000 (LIFECORE)). (A) FT-IR analysis (azide peak identified) on diHA synthesized using 60,000 mw HA; (B) FT-IR (azide peak) on diHA synthesized using 50,000 mw HA; (C) ¹³C NMR analysis of diHA (azide peak identified) synthesized using 60,000 mw HA; (D) ¹³C NMR analysis of diHA (azide peak identified) synthesized using 50,000 mw HA; (E) ¹H NMR analysis of diHA (thiol peak identified) synthesized using 60,000 mw HA; (F) ¹H NMR analysis of diHA (thiol peak identified) synthesized using 50,000 mw HA. Thiol peak in 50,000 diHA is 20%, and the one in 60,000 diHA is 16%.

FIGS. 17A-B—Mouse embryonic stem cell growth on diHA hydrogel over time in (A) proliferation media and (B) differentiation media.

FIGS. 18A-18C shows the NMR spectra of the diHA agent and the amounts of functionalization with an allyl derivative of GIKVAV (SEQ ID NO: 14) peptide of the diHA based upon the presence of the corresponding peaks in the ¹H NMR (FIG. 18A), functionalized propargyl amine (FIG. 18B), and eosin isothiocyanate with either a thiol or a methacrylate (FIG. 18C).

FIGS. 19A-19F show the functionalization of the thiol with a peptide chain (FIG. 19A), the preparation of an alkyne linked peptide for conjugation to the azide (FIG. 19B), the linking of the alkyne to the azide to obtain a second peptide sequence linked to the HA hydrogel (FIG. 19C) (In this case, both the free —SH group and the peptide linked thiol are shown as the NMR spectra indicates a mixture of the two compounds), the NMR spectra of the alkyne group after linked to the azide (FIG. 19D), and the structural comparison of the peptide modified HA hydrogen and the unmodified group (FIG. 19E), and the Young's modulus and shear of the peptide conjugated hydrogel to the unmodified hydrogel (FIG. 19F). IKVAV=SEQ ID NO: 1; CGGGERL=SEQ ID NO: 15.

FIG. 20 shows the differential diffusion and polarization of the peptide functionalized hydrogel into cells.

FIG. 21 shows the time course of HA hydrogel degradation in 100 U hyaluronidase/mL of PBS at 37° C. n=3. * indicate p-value<0.05 between formulations at the time point.

FIGS. 22A & 22B show the neural differentiated mouse embryonic stem cells (mES) in di-functional hyaluronic acid (dif HA)/methacrylate hyaluronic acid (mHA) hydrogels of various concentrations. (FIG. 22A) β3-tubulin, early stage marker of neural differentiation, staining for neural differentiated mES in various concentration of dif HA/mHA hydrogel on Day 3 and 6 (Green=β3-tubulin staining, and blue=nuclear staining), and (FIG. 22B) mRNA expression of MAP2, mature neural differentiation marker, in mES encapsulated in various ratios of dif HA/mHA hydrogel on Day 3 and 6 (n=3, * indicate p-value<0.05).

FIGS. 23A-23D show the quantification of lesion volume (FIG. 23A) and immunofluorescent staining area of ED1 (FIG. 23B), GFAP (FIG. 23C) and pan-axonal neurofilament (PanNF) (FIG. 23D) at 1 and 4 weeks after saline or 1:2 dif HA: mHA matrix injection into a spinal cord, which corresponds to 3 and 6 weeks after the initial contusion injury. (n=3, * indicate p-value<0.05).

FIG. 24 shows the immunofluorescent imaging of pan-axonal neurofilament (green) and nuclear (blue) staining in eosin tagged 1:2 dif HA:mHA matrices (red) in the lesion area one week after implantation, which corresponds to 3 weeks after injury. Scale bar=50 μm.

FIG. 25 shows the immunofluorescent imaging of eosin tagged 1:2 dif HA:mHA matrices across sequential spinal cord lesion sections (200 m between sections) at 1 and 4 weeks after matrix injection into a spinal cord, which corresponds to 3 and 6 weeks after the initial contusion injury.

FIGS. 26A-26D show the biological properties of di-functional hyaluronic acid (dif HA)/methacrylate hyaluronic acid (mHA) hydrogels composed of various mixing ratios. (FIG. 26A) Proliferation curves of HA hydrogels by MTS assay, (FIG. 26B) PAX6, early stage marker for neural differentiation, gene expression of mouse embryonic stem cells (mES) in dif HA/mHA hydrogel at Day 3 and 6 of neural differentiation (n=3). (FIG. 26C) Percentage of cells staining positive for β-tubulin III, an early stage marker for neural differentiation, in HA/mHA hydrogels at Day 3 and 6 of neural differentiation (n=5), and (FIG. 26D) β-tubulin III gene expression of mES in dif HA/mHA hydrogels at Day 3 and 6 of neural differentiation (n=3). * indicates a p<0.05.

### DESCRIPTION OF ILLUSTRATIVE EMBODIMENTS

In some aspects, the present disclosure provides hyaluronic derivatives that contain two or more linking groups, such as a thiol and/or azide-based linking group. Also provided herein are hydrogels prepared using the di-functionalized hyaluronic acid and methods of using the hydrogels to induce cell growth and stem cell differentiation. For example, in some aspects hydrogels including the hyaluronic derivatives described herein may be used to promote cell growth and/or differentiation in a neurological tissue, such as brain tissue. In some embodiments, the di-functionalized hyaluronic shows reduced stiffness and allows for flexibility in the hyaluronic backbone while introducing two different functional groups with different reaction chemistries. Additionally, in some embodiments, the functionalization of the hyaluronic acid does not degrade the hyaluronic chain. These properties render the hydrogels detailed herein very useful, for example in growth of tissue matrices or repair of damaged tissues in vivo. For example, in some aspects, the hydrogels prepared using the di-functionalized hyaluronic acid are implanted in damaged neurons with stem cells in a manner sufficient to induce stem cell differentiation and the formation of neuronal tissue.

### I. Brain Nerve Deficits and Stroke

Stroke is a leading cause of acquired disability in adults worldwide (Roger et al., 2012). Although survival after stroke has increased, little progress has been made in restoring neurological function (Group et al., 2009). Recently, stem cell therapy has emerged as a treatment with the potential to restore neurological function lost due to stroke.

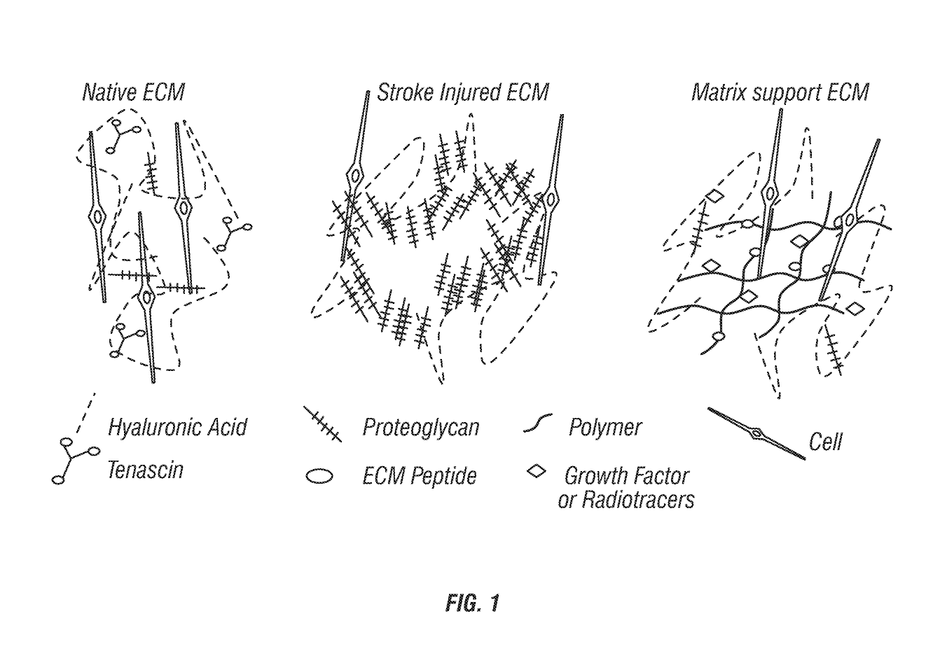

Stroke causes changes in the ECM (FIG. 1) (Al'Qteishat et al., 2006a; Al'Qteishat et al., b), which impede the ability of native NSC to migrate into the lesion and differentiate into mature neurons (Nakayama et al., 2010; Jin et al., 2003). These changes, also, create an unsupportive environment for intracerebrally implanted stem cells (Homer and Gage, 2000). aECM can mimic the chemical, physical and mechanical properties of the ECM to promote survival, adhesion, proliferation and differentiation altering the injured ECM to mitigate the barriers to axon invasion, myelination and cellular maturation. Implantation of aECM has been shown to lessen secondary injuries (FIG. 1) (Du et al., 2013; Shi, 2012; Marchand and Woerly, 1990). However, the application of aECM in the clinic has been delayed due to the necessity to utilize a combination of advance material approaches to stimulate significant restoration of neurological function and the lack of systematic studies of aECM in definitive animal models (Spector, 2014). These studies will systematically identify key material properties and synergistic interactions to enhance NSC-material interactions to improve cell survival, axon extenion and neural differentiation. This is a first step toward the development of optimized aECM for clinical use in combination with NSC therapies to lead to further advancements in the restoration of neurological function beyond those delivered by the NSC therapies alone.

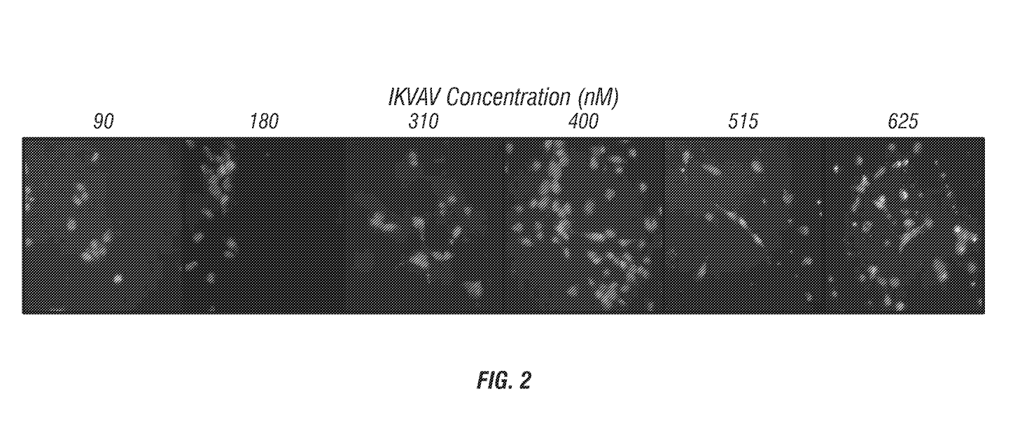

Native NSC, which migrate to the perimeter of the stroke lesion, have been found to differentiate to mature neurons, oligodendrocytes and astrocytes when isolated and cultured in vitro (Nakagomi et al., 2009). This indicates that the extracellular environment around the stoke lesion is inhibiting tissue repair. Modulating that environment to promote further migration and differentiation with an aECM will increase the restoration of neurological function beyond that achieve utilizing cells alone (Jin et al., 2010; Yasuda et al., 2010; Osanai et al., 2010). FIG. 2 demonstrates how modulating the extracellular environment with bioactive signaling peptides from laminin can have a profound effect on cellular survival and differentiation. The inclusion of synergistic bioactive signaling capable of promoting the survival and maturation of multiple neural cell types (neurons, oligodendrocytes and microvasculature) using advanced tissue engineering strategies will lead to further improvements in the restoration of neurological function and requires further investigation.

### II. Neuronal Tissue Formation and Extracellular Components

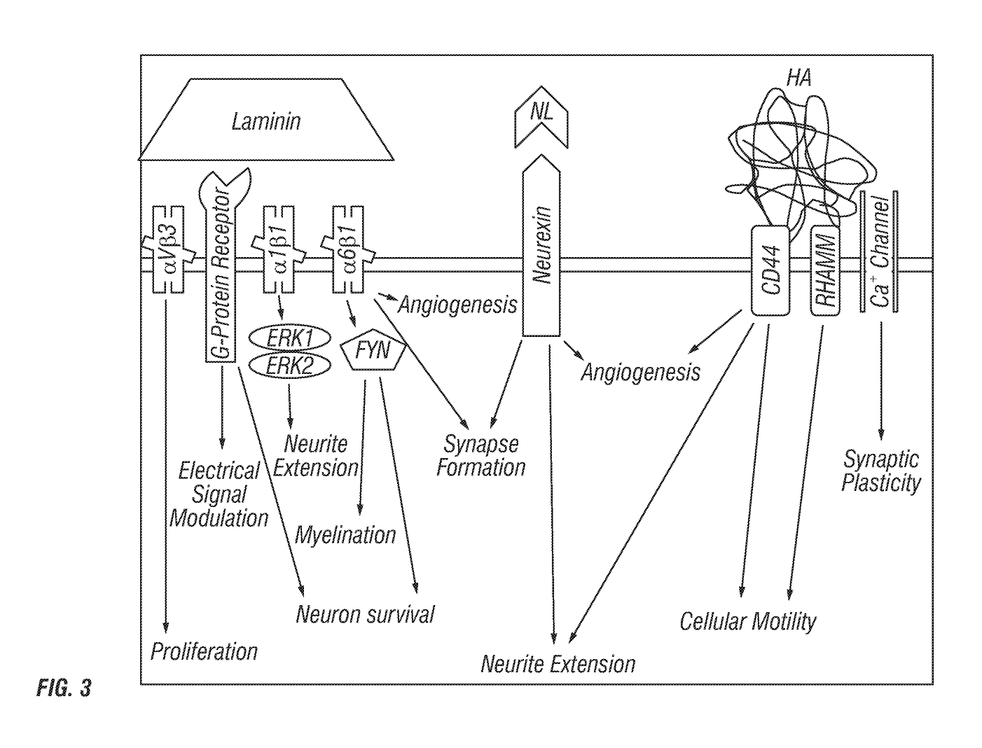

Synapse Formation and Angiogenesis in cerebral tissue involves multiple signaling pathways. FIG. 3 illustrates the contributions of select participants in cerebral tissue function, which will be discussed further in this proposal. The long term goal is to create an aECM capable of promoting the survival, axon extension, synapse formation and plasticity of native and implanted stem cells, while facilitating the integration of the graft into the tissue, promoting angiogenesis and leading to the restoration of neurological function in chronic stroke.

Laminin is a major constituent of the basal lamina, which surrounds the brain and blood vessels throughout the CNS (Timpl and Brown, 1996), and has been shown to work in conjunction with NL to promote the stabilization and maturation of newly formed neural microvasculature (Samarelli et al., 2014). Laminin, also, plays a role in synapse formation and structure (Egles et al., 2007) and has been associated with axon extension into the lesion in long term survivors of spinal cord injury (Buss et al., 2007). Other cellular effects of laminin in the CNS include increased cellular survival and proliferation, myelination, and modulation of neuronal electrical activity (Buttery, 1999; Meiners and Mercado, 2003; Liesi et al., 2001; Colognato, 2005; Garcia-Alonso and Fetter, 1996). When included in HA based aECM implanted in the brain of rats, laminin has increased neurite extension beyond that of aECM alone (Hou et al., 2005).

Neuroligin-1 (NL) is a synaptogenic adhesion protein (Ichtchenko et al., 1995; Irie et al., 1997; Sudhof, 2008), which early in development NL is expressed down the length of the neurites (Aiga et al., 2011) and promotes the survival of neurons (Schnell et al., 2014). The protein and its peptide fragments have been shown increase dendritic process formation and length and synapse formation in vivo and in vitro (Schnell et al., 2012; Gjørlund et al., 2012; Chen et al., 2012). The matrix metallopeptidase 9 cleaved fragment and the membrane tethered NL shortened bioactive fragments are biologically active both in vivo and in vitro (Gjørlund et al., 2012; Peixoto et al., 2012).

NL is widely expressed throughout the vascular system Bottos et al., 2009), and is thought to interact directly with the vascular niche participating in crosstalk between the neural and vascular system (Doetsch, 2003; Bottos et al., 2011). Although the extent of this crosstalk is not well understood, NL has been shown to promote angiogenesis through interactions with vascular endothelial growth factor A and fibroblast growth factor 2 (Bottos et al., 2009; Rissone et al., 2012) and promote vessel stabilization and maturation through interactions with α6β1 integrin and laminin Samarell et al., 2014).

Hyaluronic Acid (HA) is a high molecular weight glycosaminoglycan present in the ECM of the developing brain in higher amounts than the adult brain, but even at lower amounts is still major constituent in the adult ECM (Margolis et al., 1975; Dityatev et al., 2010). HA has multiple modification sites that can be modified by several methods to form a hydrogel and has been used extensively as a scaffolding base in tissue engineering for numerous tissues (Fujiwara et al., 2000; Prestwich et al., 1998; Gamini et al., 2002; Slaughter et al., 2009). HA hydrogels have been shown to be capable of supporting NSC growth and differentiation in vitro (Pan et al., 2009). When implanted in rat brains, HA hydrogels have increased cell infiltration, angiogenesis and integration with host tissue compared to control (Hou et al., 2005). HA in the perisynaptic ECM has been implicated in synapse plasticity through modulation of calcium channels (Kochlamazashvili et al., 2010).

### III. Hyaluronic Acid Derivatives

The hyaluronic acid derivatives provided by the present disclosure are shown, for example, above in the summary of the invention section and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Hyaluronic acid derivatives of the invention may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

Hyaluronic acid derivatives of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the hyaluronic acid derivatives of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### IV. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “-” means a single bond, “=” means a double bond, and “≡” means triple bond. The symbol “----” represents an optional bond, which if present is either single or double. The symbol “ [symbol] ” represents a single bond or a double bond. Thus, for example, the formula

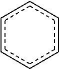

includes

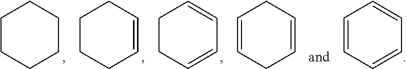

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

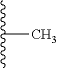

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

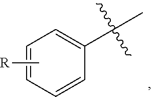

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

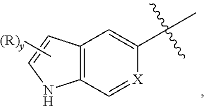

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” when used without the “substituted” modifier refers to a divalent unsaturated aliphatic group, with two carbon atoms as points of attachment, a linear or branched, a linear or branched acyclic structure, at least one nonaromatic carbon-carbon triple bond, no carbon-carbon double bonds, and no atoms other than carbon and hydrogen. The groups —C≡C—, —C≡CCH₂—, and —CH₂C≡CCH₂— are non-limiting examples of alkynediyl groups. It is noted that while the alkynediyl group is aliphatic, once connected at both ends, this group is not precluded from forming part of an aromatic structure. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

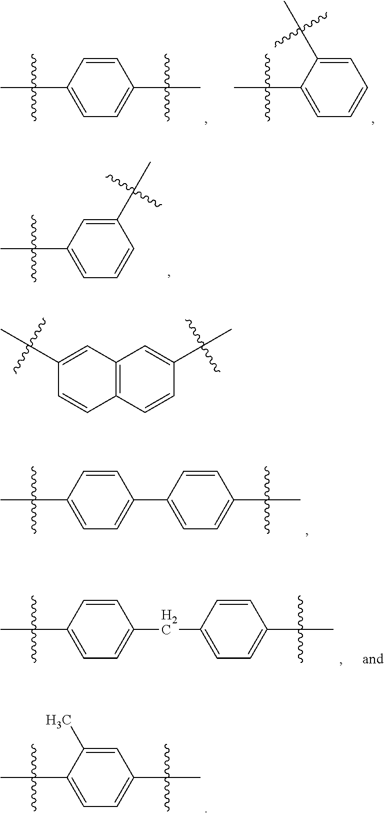

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected hydroxy group is a group of the formula PG_{H}O— wherein PG_{H} is a hydroxyl protecting group as described above.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

In some embodiments, provided di-functional HA can act as linker molecule with broad application to biomedicine and tissue engineering. Examples of such applications include, but are not limited to, as a multi-functional scaffold base material for neural tissue engineering applications, as a replacement or supplement to those tissues that are largely composed of hyaluronic acid, such as but not limited to cartilage and thus diHA has many uses in orthopedic medicine including, but not limited to a role as an injectable lubricant. In additional embodiments, diHA provides a broadly applicable tissue engineering matrix that could be injected and form the final matrix within the body, for example as dermal filler useful for reconstructive and cosmetic applications. In other embodiments, diHA can also be used as a therapeutic delivery agent in non-tissue engineering applications. In still other embodiments, diHA also provides a method to increase the persistence of additional signal or therapeutic molecules (drugs, bioactive signals, proteins) in injections.

In some embodiment the diHA describe herein, utilizes a single functional chemistry for tethering and gelation, the addition of a second chemistry increases the total concentration and control of tether and gelation. The thiol and azide chemistries do not interact with each other meaning functionalization of one chemistry dose on reduce the availability of the other chemistry to interact with additional bioactive signals or gelation chemistries. Functionalization based on classical reactions used in polysaccharide chemistry typically have unsatisfactory efficiency and harsh conditions leading to polymer chain degradation or was not cleaved during the synthesis (Mergy et al., 2012), the current diHA method does not have these disadvantages. Sufficient functionalization efficiency for hydrogel formation is achieved without HA backbone degradation. Functionalization of HA polymer backbone has also been associated with chain stiffing, which reduces the ability of the material to interact with cells reducing attachment (Camci-Unal et al., 2013). DiHA allows for the addition of two functional chemistries which maintaining enough flexibility in the HA backbone to allow for cellular attachment and differentiation. Thus a surprising and unexpected aspect was that two functional chemistries could be put on one backbone without breaking the polymer backbone and that the backbone remained flexible enough to allow cellular attachment and differentiation without further functionalization.

In some embodiments, provided is one-pot synthesis of diHA, a strategy that improves efficiency of the chemical reaction by subjecting to successive chemical reactions in just one reactor, thus avoiding a lengthy separation process and purification of the intermediate chemical compounds, saving time and resources while increasing chemical yield.

### Example 1—Synthesis of Difunctional HA

Synthesis of Di-Functional HA

Hyaluronic acid (0.04 g, M_{W}=60,000 kDa) was dissolved in 8 mL ultrapure water (DDW) (0.5% w/v solution). The pH of the solution was raised to 10 with 1M NaOH. A two-folder molar excess of ethylene sulfide was added to the HA solution very slowly and the reaction mixture was stirred overnight in the chemical hood at room temperature. The solution was filtered through 3-cm bed of Celite® 545. A five-fold molar excess of DTT was added to filtered solution, and raised a pH of 8.5 with 1M NaOH. The reaction was stirred overnight in chemical hood at room temperature. 78.4 mg of MES (2-(N-morpholino)-ethanesulfonic acid buffer) (50 mM, pH 4), 95.6 mg of EDC-HCl, 57.4 mg of NHS ((N-hydroxysuccinimide), and 154 μL of 11-azido-3,6,9-trioxaundecan-1-amine were added on 8 mL of HA-sulfhydryl solution, and the reaction of mixture was stirred 24 hrs in the chemical hood at room temperature. The solution was dialyzed (cutoff=12-14 kDa) against 1M NaCl for 1 day, and then an additional DDW for 5 days. DW was changed every day. The solution was lyophilized to obtain HA-SH-AA (di-functional HA) powder for use in hydrogel formation.

The step-by-step process is shown below in Table 1.

TABLE 1
Step by Step Process for Formation of Di-Functionalized Hyaluronic Acid

A. HASH (HA-sulfhydryl)
1. 0.04 g (60k sodium HA) + 8 ml DW (0.5% HA in DW)
2. pH 10 using 1M NaOH
3. Add 192ul ES/8 ml HA solution (5-fold molar excess ethylene sulfide)
slowly - dropwise 1 drop/2-3 min
4. Reaction overnight at RT under chemical hood
5. Filter 3 cm bed of Celite ® 545
6. Add 0.1M stock solution 32ul/8 ml HA + ES (5-fold DTT)
7. pH 8.5 using 1M NaOH
6. Reaction overnight (24 hrs) at RT under chemical hood
7. pH 3.5 using 6N HCl
Optionally, dialysis with a 10K membrane was then conducted for 5 days
(everyday DW change), and then lyophilizer (power), however this was
only in the case that reaction should be terminated with only the
thiol modification.
B. HAAA
8. 0.04 g HA/ES/DTT/8 ml DW + 0.0784 g MES (50 mM, pH 4)
9. Add 0.0956 g EDC-HCl/154ul AA/0.0574 g NHS
10. Reaction 24 hrs at RT
11. Dialysis (12 kDa) at NaCl for 1 day
12. Dialysis (12 kDa) at DW for 5 days (everyday water change)
13. Freeze
14. Dry using lyophilizer
15. Gelling test and structure analysis using by analytical techniques such
as nuclear magnetic resonance, mass, and infrared spectroscopy.
(HA = hyaluronic acid; DW = ultrapure water; ES = ethylene sulfide; DTT = dithiothreitol; MES = 2-morpholinoethanesulfonic acid; EDC-HCl = N-(3-Dimethylaminopropyl)-N′-ethylcarbodiimide hydrochloride; NHS = N-hydroxysuccinimide; AA = 11-azido-3,6,9-trioxaundecan-1-amine)

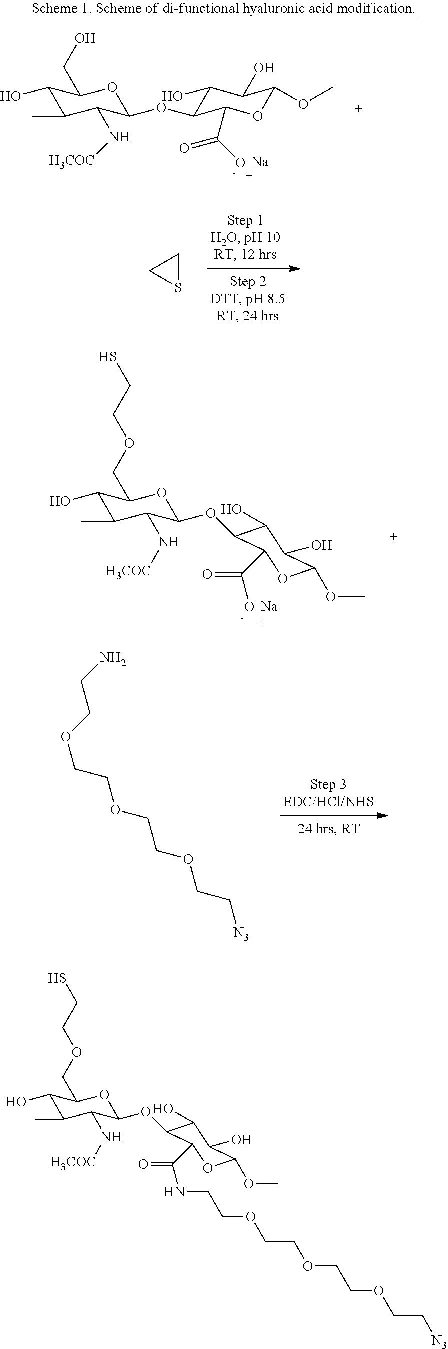

### Example 2—Optimal Concentrations of Bioactive Peptides

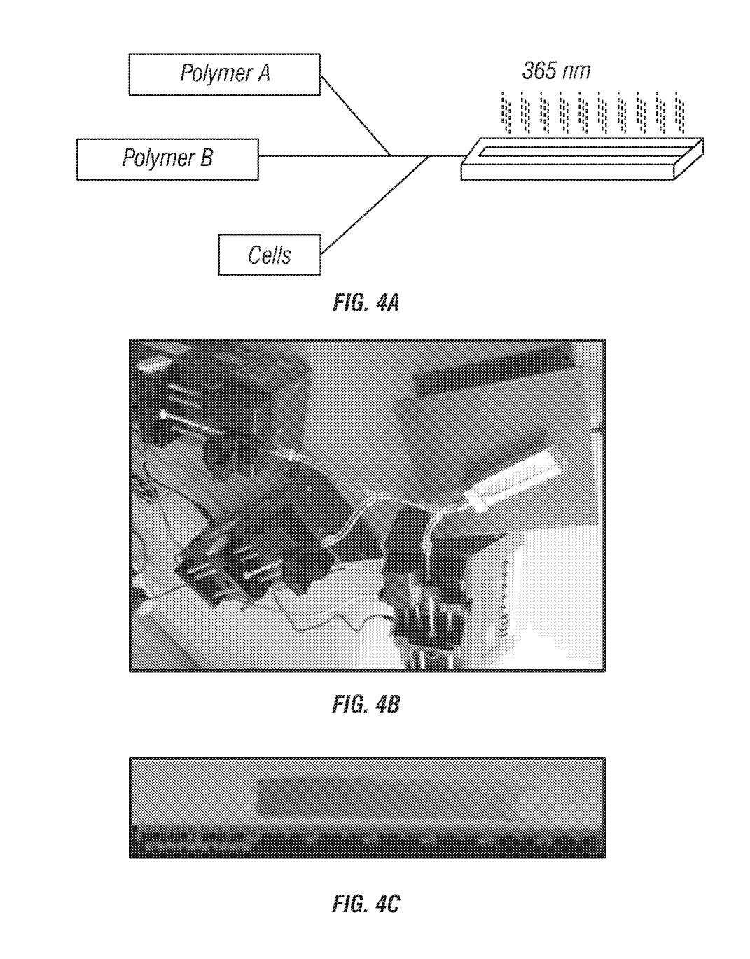

To individually determine the optimal concentration of bioactive peptides, such as but not limited to, laminin and neuroligin-1 (NL) needed to increase hiPSC derived NSC survival, axon extension, and synapse formation continuous gradient samples are used for two and three dimensional in vitro culture. Laminin and NL have not been widely studied in an artificial extracellular matrix (aECM) for the brain. The therapeutic concentrations and combinations of peptide fragments are not known. Previously, the inventors have utilized a continuous gradient pump system (FIG. 4) to examine optimal peptide concentration for orthopedic applications (Smith Callahan et al., 2013a; Smith Callahan et al., 2013b). This study applies the same skills to the CNS in order to determine the optimal concentration of tethered bioactive fragments from laminin and NL to increase hiPSC derived NSC survival, axon extension and synapse formation.

Peptides from laminin and NL (Table 2) will be synthesized using solid phase Fmoc chemistry and will be coupled with methacryloyl chloride (Johnson et al., 2010). Peptide concentration gradients will be made in a manner similar to previously described using the gradient system in FIGS. 4A-C(Smith Callahan et al., 2013a; Smith Callahan et al., 2013b). Hydrogels will be characterized as previously described (Liesi et al., 2001). Briefly, Young's and shear modulus will be determined with a TA.TX texture analyzer; swelling studies of size and weight is used to determine swelling ratio, mesh size and water content; total peptide concentration will be determined using a Lowry assay; and the bioavailable peptide concentration is determined with appropriate antibody if available or a biotinylated lysine will be added to the N-terminus of the peptide and then detected with a streptavidin secondary antibody after hydrogel fabrication.

TABLE 2
Bioactive peptide fragments from laminin and neuroligin-1.

| Protein | Bioactive Signaling Peptides | Source | SEQ ID NO: |
|---|---|---|---|
| Laminin | IKVAV | J Biol Chem. 1989; 264(27): 16174-82. | 1 |
| | YIGSR | Biochemistry. 1987; 26(22): 6896-900. | 2 |
| | RNIAEIIKDI | ACS Chemical Biology, 2007. 2(5): 347-55. | 3 |
| neuroligin-1 | SEGNRWSNSTKGLFQRA | FASEB J 2012 26 (10): 4174-86. | 4 |
| | LRE | Neuron. 2007 56(6): 979-91. | 5 |

For cell experiments, hiPSC derived NSC will be encapsulated in the peptide gradient hydrogels (Smith Callahan et al., 2013a; Smith Callahan et al., 2013b). Cell laden hydrogels are maintained in NSC maintenance media (DMEM:F12 containing 1×N2, 1×B27, 20 ng/mL bFGF, 2 μg/mL heparin, 2 mM Glutamax™, 1% non-essential amino acid, and 55 mM 2-mercaptoethanol) in order to support hiPSC derived NSC survival, but permit an assessment of biological effect of the peptides due to the lack of cytokines driving further differentiation in the media. Over a 8 week time course, cell survival and differentiation will be assessed as described in Table 3 according to previously published protocols (see Smith Callahan et al., 2013c; Liu et al., 2002).

TABLE 3
Strategy for testing neural stem cell response to aECM. Each gradient
sample will be divided into at least 5 discrete samples for analysis.

| | Groups | |
|---|---|---|
| | | Gradient |
| | Control: Tissue | Samples: laminin peptide, & |
| Analysis | Culture Plastic | neuroligin-1 peptide |

| | |
|---|---|
| Cell Survival | DNA Content (Horechest dye) & Tunnel Staining |
| Differentiation | Immunofluorescence: quantification of TUJ1 |
| | espressing neurite lengths & Percent of cells |
| | expressing TUJ1, NeuN & synapsin. qPCR: Pax6, |
| | TUJ1, MAP2, NeuN, NF & synapsin. |

All statistical analysis will be performed using one-way analysis of variance and a comparison-wise Tukey test at 95% confidence. Cell culture experiments will be conducted twice using quadruplicate samples.

It is expected that peptides at specific concentrations will augment NSC survival, axon extension and synapse formation. However it is possible that the selected peptides do not produce the desired effects in hiPSC derived NSC. In that case, additional media supplements will be added to facilitate differentiation, longer linkers will be used to tether peptides to limit steric hindrance or additional peptides from laminin and NL will be examined in the gradients.

### Example 3—Hyaluronic Acid Matrices

Hyaluronic acid matrices capable of presenting bioactive peptides from laminin and neuroligin-1 at independent concentrations will be fabricated and characterized. The presentation of multiple signaling elements in an aECM will provide synergistic signaling to overcome the inhibitory signals in the CNS and facilitate the reestablishment of synaptic connections in the lesion. Laminin signaling will facilitate the extension of cells into CNS lesions (Buss et al., 2007). When combined with NL and HA, the advanced aECM proposed here will expedite the restoration of neurological function through increased survival, axon extension and synapse formation.

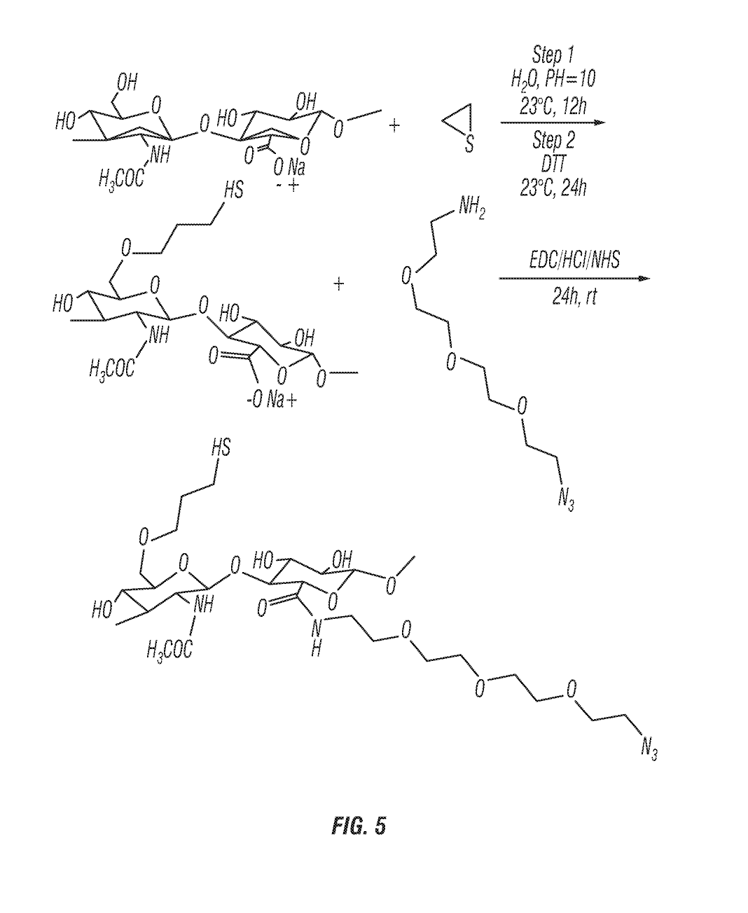

High molecular weight HA (˜75 kDA) will be modified with a thiol and azide group as described in FIG. 5 (Serban et al., 2008; Crescenzi et al., 2007). Peptides will be synthesized in a similar manner as in example 2 and be coupled with methacryloyl chloride, 5-hexynoic acid, or cyclooctyne (Johnson et al., 2010; Lin et al., 2013). Peptides will be crosslinked to HA in triethanolamine-buffered saline containing CuCl at 37° C. for 1 hr (Crescenzi et al., 2007; Lin et al., 2013). Throughout the synthetic process HA solutions will be purified by forced air dialysis and ¹H NMR and MALDI-TOF MS will be used determine modification efficiency.

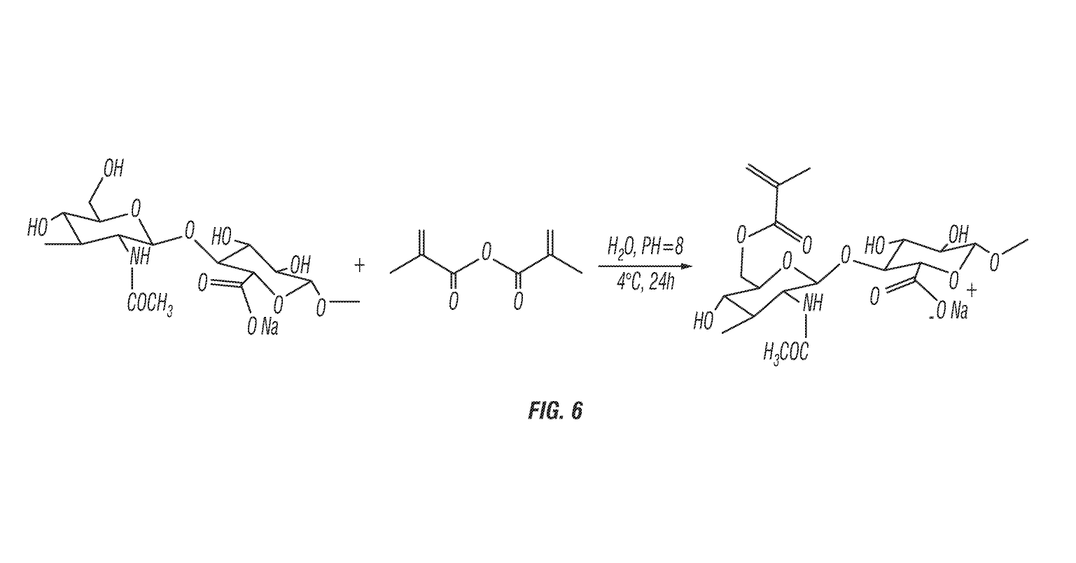

For gelation via Michael addition, high molecular weight HA will methacrylated as described in FIG. 6 (Burdick et al., 2005; Smeds and Grinstaff, 2001). An equal ratio of available thiol to methacrylate in the HA will be used to form the aECM. The aECM will then be characterized as described in example 2.

It is expected that HA will remain biologically active following both the chemical derivation and polymerization. If the extent of chemical derivation is low or the biological activity of the peptides is significantly tempered in the hydrogel, alternative strategies will be pursued including alternative purifications and inclusion of longer linkage segments to attach the peptide to the backbone. Additionally, the examination of the oligodendrocyte response and microvasculature invasion in the aECM, may direct further modification of the backbone to tri-functionalization, or development of hybrid matrices using additional polymers and peptides to further emulate aspects of the native brain ECM.

### Example 4—Evaluate hiPSC Derived NSC Survival, Axon Extension and Synapse Formation

The hiPSC derived NSC survival, axon extension and synapse formation in hyaluronic acid matrices established using the bioactive peptides from laminin and neuroligin-1 in vivo will be evaluated. To establish the safety and efficacy of therapies that use such advanced biomaterials either alone or in combination with cell therapy in an animal stroke model in order to develop the combination therapies necessary to generate clinical functional recovery, more studies must be done. The inventors will implant the optimized matrices developed using the methods described herein in rodent models to establish their value in improving hiPSC survival and maturation beyond that of the standard stem cell therapy procedures.

A moderate transient focal ischemia is induced in all SCID mice by right middle cerebral artery occlusion (MCAO) using the intraluminal thread method as previously described (Li et al., 2010). Seven days after the MCAO, a neurological deficit score (Table 4) will be determined and behavioral testing (Table 5) will be conducted as previously described (see for example, Hatcher et al., 2002; Hua et al., 2002; Cao et al., 2010; Xiong et al., 2011). Mice are assigned to transplant groups (Table 5) and ˜200,000 GFP+hiPSC derived NSC will be mixed in with 10 μL of saline or HA solutions from Example 3 with and without peptides will be injected into the infarcted striatum and cortex. Behavioral testing will be conducted weekly for 4 weeks (Table 5). After the final behavior test, the mice in each group will subdivided into 3 groups. Subgroup 1 (containing 5 mice) will be stained with triphenyltetrazolium chloride to quantify the infract volume. Subgroup 2 (containing 5 mice) will be used for Evans blue extravasation to examine the integrity of the blood brain barrier via spectrometry. The 3rd subgroup (containing 6 mice) will be used for cresyl violet staining to identify the infarct and immunofluorescence (Table 5).

TABLE 4
Neurological Deficit Scoring System.

| Score | Neurological Deficit |
|---|---|
| 0 | no deficit |
| 1 | flexion of contralateral torso and forlimb |
| | upon lisfting of the whole animal by tail |
| 2 | circling to the contralateral side, when |
| | helped by tail with feet on floor |
| 3 | spontaneous circling to the contralateral |
| | side |
| 4 | no spontaneous motor acitivity |

TABLE 5
In vivo aECM Biological Testing.

| | Groups | | |
|---|---|---|---|
| | | | NSC + HA + |
| Analysis | NSC | NSC + HA | Laminin + NL |

| | |
|---|---|
| Survival | Total number of GFP⁺ NSC |
| Proliferation | % of GFP⁺ and Ki67⁺ NSC |
| Differentiation | length of TUJ1/GFP neurites, % |
| | of TUJ1/GFP, % of NeuN/GFP & |
| | % of synapsin/GFP |
| Behavioral | Neurological Deficit Score, |
| Testing | Rotarod Test, Corner Turning |
| | Test & SmartCage System |

A priori power analysis using G*Power 3.1.9 software with a statistical power of 0.8 (α error<0.05) indicates 45 animals will be necessary for the study based on the effect size of a previously published dataset (Jin et al., 2010). All statistical analysis of collected data will be performed using one-way analysis of variance and a comparison-wise Tukey test at 95% confidence.

The HA matrices with multiple signaling peptides are anticipated to provide superior microenvironments for hiPSC derived NSC survival, axon extension and synapse formation compared to hydrogels containing lesser or no peptides signals. If this fail, strategies such as those described in Examples 2 and 3 will be utilized to further optimize the aECM. Should the integration of human cells in animal models pose a problem, mouse NSC will be differentiated from mouse iPSCs. Typically, optimal conditions for human stem cells cultured on matrices that have worked for similar rodent stem cells with little alteration (Smith et al., 2010; Smith et al., 2009), so if a change in species be necessary to facilitate animal studies it should not pose a major challenge to progress.

### Example 5—Characterize Spatial Location of Di-Functional Hyaluronic Acid within Gradient Polyethylene Glycol Hydrogels by Radioactive Detection

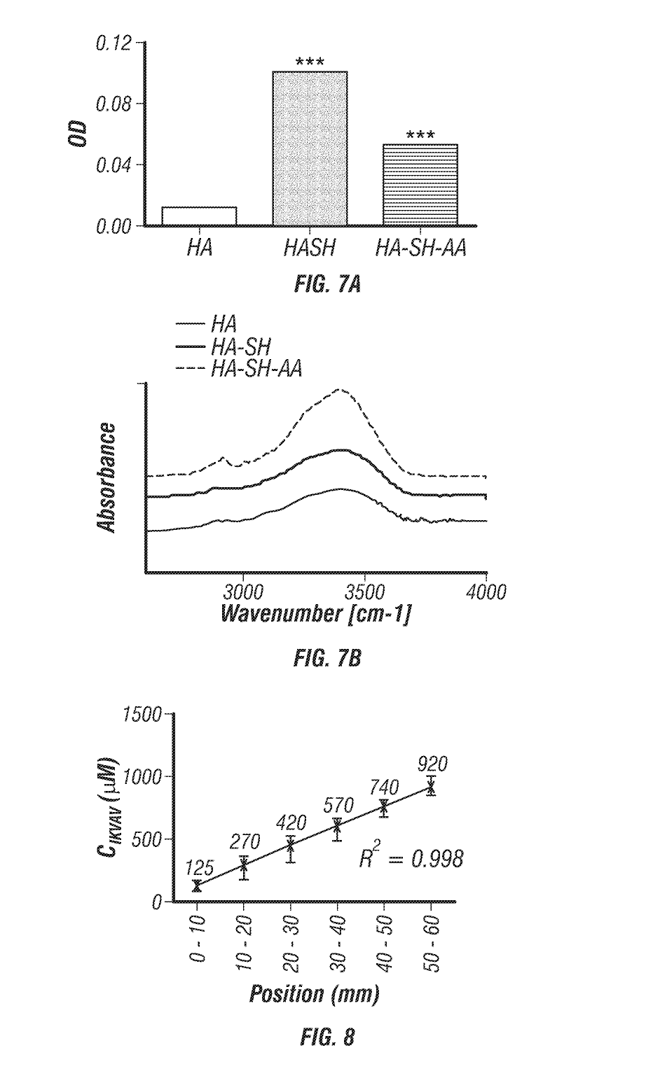

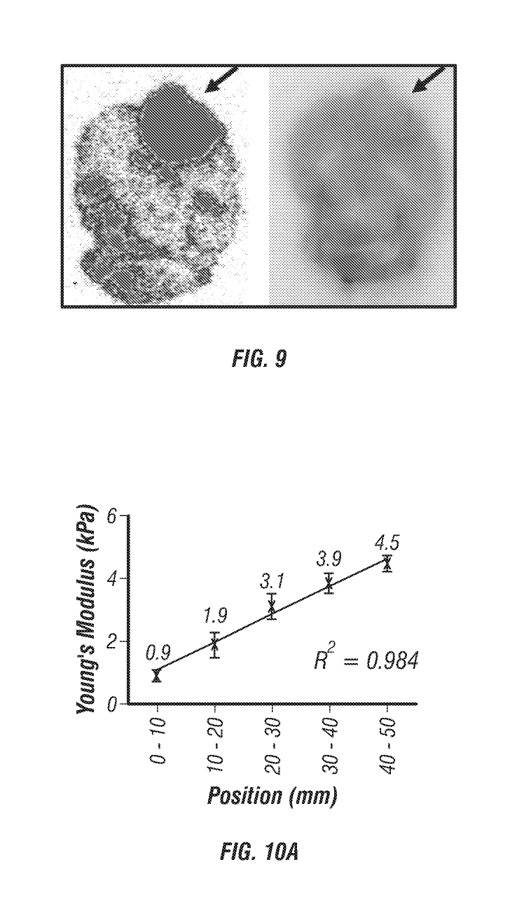

The concentrations and spatial location of bioactive signal and polymer content are rarely quantified in tissue engineering and device coating applications and when done, fluorescent or colorimetric readouts are typically used, which provide little information about the spatial location of these signaling and structural elements in the 3D culture. (Zustiak et al. 2010, Smith Callahan, Ganios, et al. 2013). Utilizing radiotracers enables quantitative analysis of HA components and significantly improves both the determination of concentrations and spatial locations beyond currently available assays. HA functionalized with reactive moieties for Michael addition and click chemistry as developed by the inventors (FIGS. 5 and 7A-B) is used to create a HA gradient within polyethylene glycol hydrogels as has previously been done with other bioactive signaling elements by the inventors (FIGS. 4A-C and 8). Radiolabeling methods similar to those described in Ghosh et al. 2013 and Ghosh et al. 2015 (incorporated herein by reference, see also FIG. 9) are then used and compared to standard flourometric and colorimetric readouts to determine the concentration and spatial location of the HA within the matrix. Positron-emitters, Gallium-68 (⁶⁸Ga, t½=68 min) and Copper-64 (⁶⁴Cu; t½=12.7 hr), will be used due to their high sensitivity (e.g., fM-pM concentrations) as radiolabels. Both radiometals have been clinically used for Positron Emission Tomography (PET) in cancer.

High molecular weight HA (˜75 kDA) is modified with a thiol and azide group as shown in FIG. 5 (Crescenzi et al. 2007, Serban et al. 2008). Throughout the synthetic process HA solutions are purified by forced air dialysis. ¹H NMR, ¹³C NMR and MALDI-TOF MS is used determine modification efficiency. BFCA 1,4,7,10-tetraazacyclododecane-N,N′,N″,N″′-tetraacetic acid (DOTA) based ⁶⁸Ga (t½=68 min) and ⁶⁴Cu (t½=12.7 h) radio tracers coupled to dibenzocyclooctyne will be synthesized in a similar method to that previously described in Ghosh et al. 2013. Throughout the synthesis process reaction efficiency will be monitored by ¹H NMR, ¹³C NMR and ESI MS.

Modified HA is then be dissolved in 10% polyethylene glycol dimethacrylate (PEGDM). HA gradient is then made in a manner similar to previous described using the computer controlled gradient making system in FIGS. 4A-C and 8 (Smith Callahan, Policastro, et al. 2013). The HA backbone will then be spatially located and quantified using the Alcian Blue Method (Frazier et al. 2008, Smith Callahan, Ganios, et al. 2013). Azides functionalized to the HA backbone are conjugated with dibenzocyclooctyne-functionalized chelating agents to allow radiometal incorporation, or with a dibenzocyclooctyne-functionalized fluorescent probe as previously described using copper-fee click chemistry (Zheng et al. 2013). To maximize spatial resolution, the location of the radiolabels will be detected using a radiometric phosphor imager that provides quantitative, filmless autoradiography (FIG. 9) (Yang et al. 2004), while fluorescent probes will be quantified using a Tecan® Infinite M1000 and spatially located using a Nikon® TE2000-E inverted microscope. Results are compared to ¹H NMR and ¹³C NMR results for detection of functionalization and Alcian Blue Method for spatial location to examine accuracy of specific methods.

If the current does not allow for greater sensitivity in spatial and moiety characterization than currently utilized fluorometric and colorimetric assays, the radiotracers will be bound to the HA backbone prior to matrix formation, the radiotracer concentration will be increased, or alternative chemistries will be utilized for coupling of labels, near-infrared fluorescence detection agent will be added to radiotracer, and/or HA concentration within gradient will be altered further to determine the assays range of sensitivity.

### Example 6—Determine Effects on Radiotracer Binding on Hyaluronic Acid Matrix Degradation, Mechanics and Material Properties

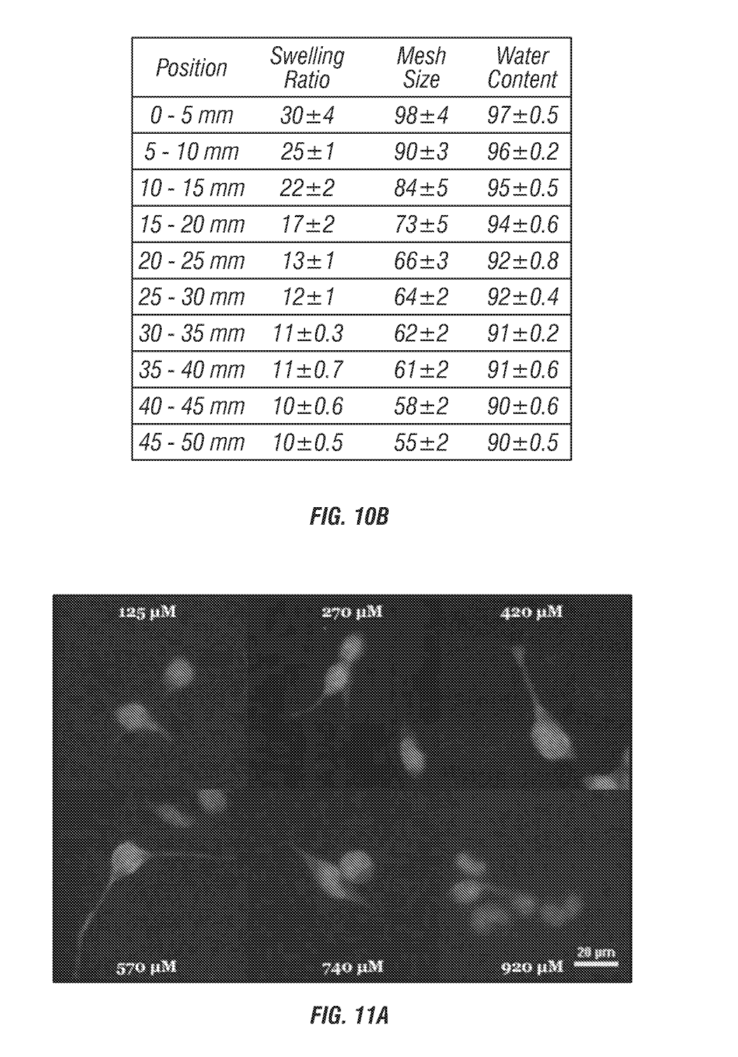

Modification of HA backbone has been shown to alter the degradation rate, mechanical properties and material properties of HA matrices (Baier Leach et al. 2003). Previously described methods of hydrogel characterization (see FIGS. 8 and 10A-B) are utilized to determine the effects of radiotracer binding on the properties of the HA matrix (Smith Callahan, Ganios, et al. 2013, Smith Callahan, Policastro, et al. 2013d). Alterations in these properties also have the potential to effect the longevity of HA matrix, ability of the HA matrix to support cellular survival and differentiation, and the eventual clearance of the HA matrix during future in vivo applications and should be characterized in early development to determine the suitability of the technology for clinical use.

Modified HA with and without coupled radiotracers are mixed with methacrylated HA at concentrations that contain an equal ratio of available thiol to methacrylate and allowed to form a gel overnight via Michael addition. For degradation studies, samples will be incubated in PBS with 2.6 u/mL hyaluronidase at 37° C. for 1 week (Khetan et al. 2009). Supernatant will be collected daily and the amount of uronic acid, a degradation product of HA, in the supernatant will be quantified using the carbazole reaction technique (Bergman et al. 1970). A gamma counter will also be used to determine the amount of radioactivity in the supernatant to further quantify degradation. The Young's and shear modulus are determined with a TA.TX texture analyzer as previously described, while material properties (mesh size, swelling ratio, and water content) are determined as previously described in Baier Leach et al., 2003, and Smith Callahan, Policastro et al. 2013 (incorporated herein by reference).

Methods of material characterization utilized in this example are well established. If problems arise, alternative methods, which are equally established, will be used. If uronic acid assay output does not yield a clear picture of degradation, the mass loss of the matrices can also be tracked (Baier Leach et al. 2003). For mechanical testing, dynamic mechanical analysis or rheological methods will be utilized (Khetan et al. 2009, Holloway et al. 2014).

### Example 7—Evaluating Ability of Radiotracers to Spatially Locate Di-Functional Hyaluronic Acid in Three Dimensional Cultures of Human Induced Pluripotent Stem Cell Derived Neural Stem Cells and the Effects of the Radiotracers on Cellular Survival, Neurite Extension, and the Gene Expression of Mature Markers of Neural Differentiation

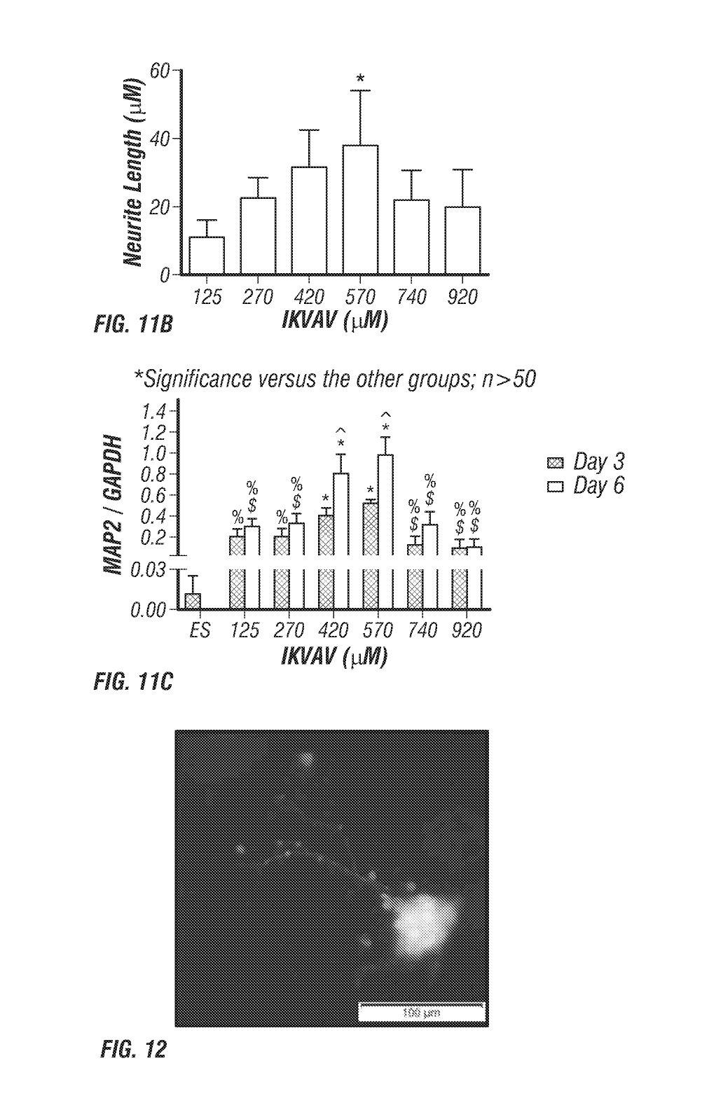

To improve cell-modified HA interaction, additional bioactive signaling proteins are coupled to HA matrixes (Shu et al. 2004). Bioactive peptide fragments have similar effects on cellular behavior as proteins, but are easier to tether with regio- and chemo-selectivity and spatially disburse in 3D materials (Meiners et al. 2003). The fabrication of bioactive peptides containing matrices for human cell culture has been previously described (Smith Callahan, Ma, et al. 2013, Smith Callahan, Policastro, et al. 2013). A bioactive peptide fragment from laminin, IKVAV (SEQ ID NO:1), was selected for this study with the radiotracers due to the ability to promote cellular survival and proliferation, neurite guidance, myelination, and the modulation of neuronal electrical activity (FIGS. 11A-C) (Garcia-Alonso et al. 1996, Buttery et al. 1999, Colognato et al. 2005). Preliminary data using clinically relevant hiPSC derived NSC indicates that the IKVAV (SEQ ID NO:1) peptide coupled within a 3D matrix can support survival and neurite extension (FIG. 12), allowing the examination of changes in cellular behavior and attachment to the matrix caused by the inclusion of the radiotracers.

Laminin peptide, IKVAV (SEQ ID NO:1), is synthesized using standard Fmoc chemistry and coupled to methacryloyl chrloride (Elbert et al. 2001). A percentage of free thiols on the HA backbone will be consumed to couple the IKVAV (SEQ ID NO: 1) peptide to the backbone. The remainder of the free thiols are used to form the matrices described in Example 6.

NSC derived from the HDF51 hiPSC line will be encapsulated in HA matrices containing IKVAV (SEQ ID NO:1) with and without the radiotracers. NSC derived from the HDF51 hiPSC line on matrigel coated tissue culture plastic will be used as a control. Each sample will be cultured individually in wells of a 48 well plate for up to 2 weeks in DMEM:F12 media containing 1% Glutamax™, 1% non-essential amino acids, 0.5% N2 supplement, 1% B27 supplement, 20 ng/mL brain-derived neurotrophic factor, 20 ng/mL glial cell-derived neurotrophic factor, 200 ng/mL ascorbic acid, and 1 μM cAMP. After 3, 9, and 14 days, cellular survival, axon extension, and gene expression will be examined as described Example 2 (see Table 3 above) according to previously published protocols (Liu et al. 2002, Smith Callahan, Ma, et al. 2013).

All statistical analysis will be performed using one-way analysis of variance and a comparison-wise Tukey test at 95% confidence. Cell culture experiments will be conducted twice using quadruplicate samples.

It is expected that IKVAV (SEQ ID NO:1) will remain bioactive and radiotracers will not have significant effect on cellular behavior in long term culture. Should the incorporated IKVAV (SEQ ID NO:1) concentration not adequately support cellular attachment and survival longer linkers will be used to tether peptides to limit steric hindrance, the concentration of IKVAV (SEQ ID NO:1) is increased or additional bioactive peptides from laminin (YIGSR (SEQ ID NO:2)) is added to the matrix. If radiotracers inhibit cell material-interface longer, flexible linkers will be used, the radiotracer concentration will be reduced, or an alternative detection agent will be utilized.

### Example 8—Preliminary Results

Molecular structure of di-functional (diHA) and methacrylate HA (mHA) are shown in FIGS. 5 and 6. The thiol group was characterized by ¹H NMR (Bruker 600 MHz, Deuterium oxide (D₂O), colorimetric 2,4,6-trinitrobenzene sulfonic acid (TNBS) assay. The azide group was characterized by ¹³C NMR (Bruker 600 MHz, D₂O), Fourier transform infrared spectroscopy (FT-IR) spectrum, and isotopic dilution experiment with ⁶⁸Ga. Chelating reagent with ⁶⁸Ga used to cap azide in HA backbone, radioTLC peaks used to calculate the average number of chelating moieties that capped the azide functional groups. diHA and methacrylate HA (mHA) were used to fabricate hydrogels using photopolymerization with Irgacure 2959 photoinitiator and 2.3 mJ cm⁻² UVA light for 5 min. Hydrogels of varying composition ranging from 1:2 and 1:5, diHA: mHA were tested for swelling ratio, water contents, and mechanical properties, young's and shear modulus. D3 mouse embryo stem cells (mES) were seeded in HA hydrogel in 3D system, yielding a final cell encapsulation density of 1×10⁶ cells/mL. mES in 3D HA hydrogels induced neural lineage on neural differentiation media containing 80% F-12, 20% neurobasal medium, 1% penicillin/streptomycin, 0.8×N2, 0.2×B27, 10 mM sodium pyruvate, and 2 μM retinoic acid. After 3 days, neural differentiated mES were examined by β-tubulin III staining, and count staining on nuclei was using 4′,6-diamidino-2-phenylindole (DAPI). Images are taking by confocal microscope, and counted β-tubulin III positive cells.

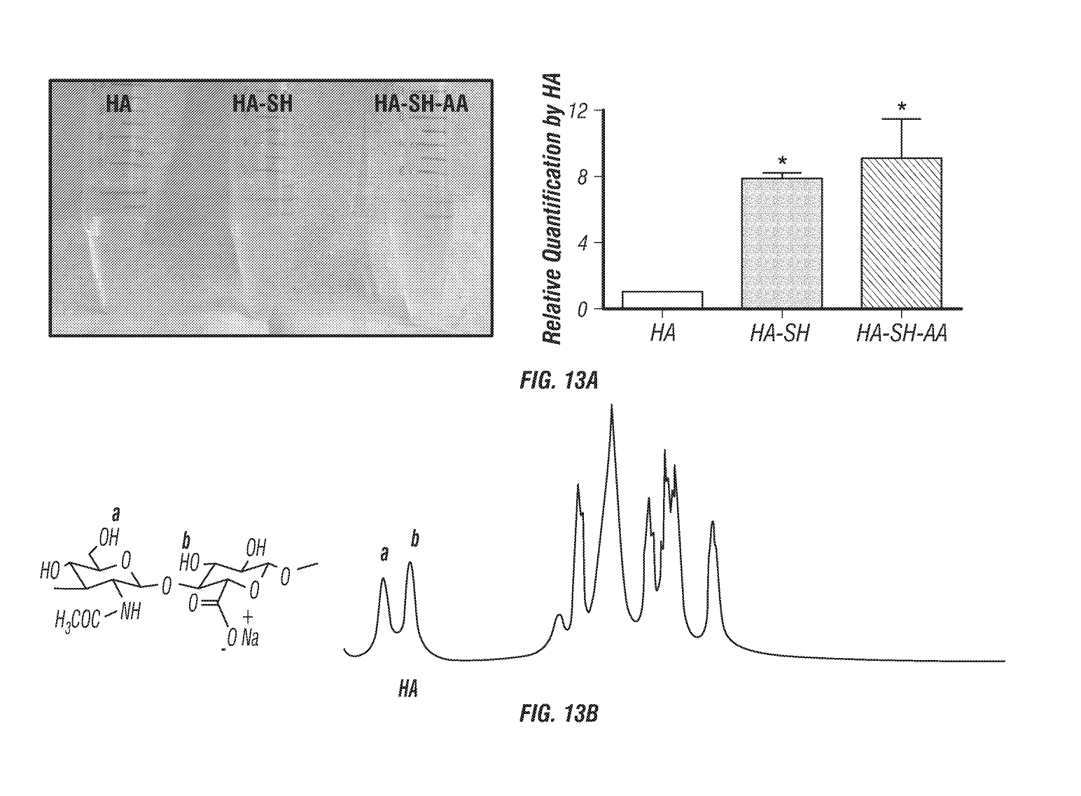

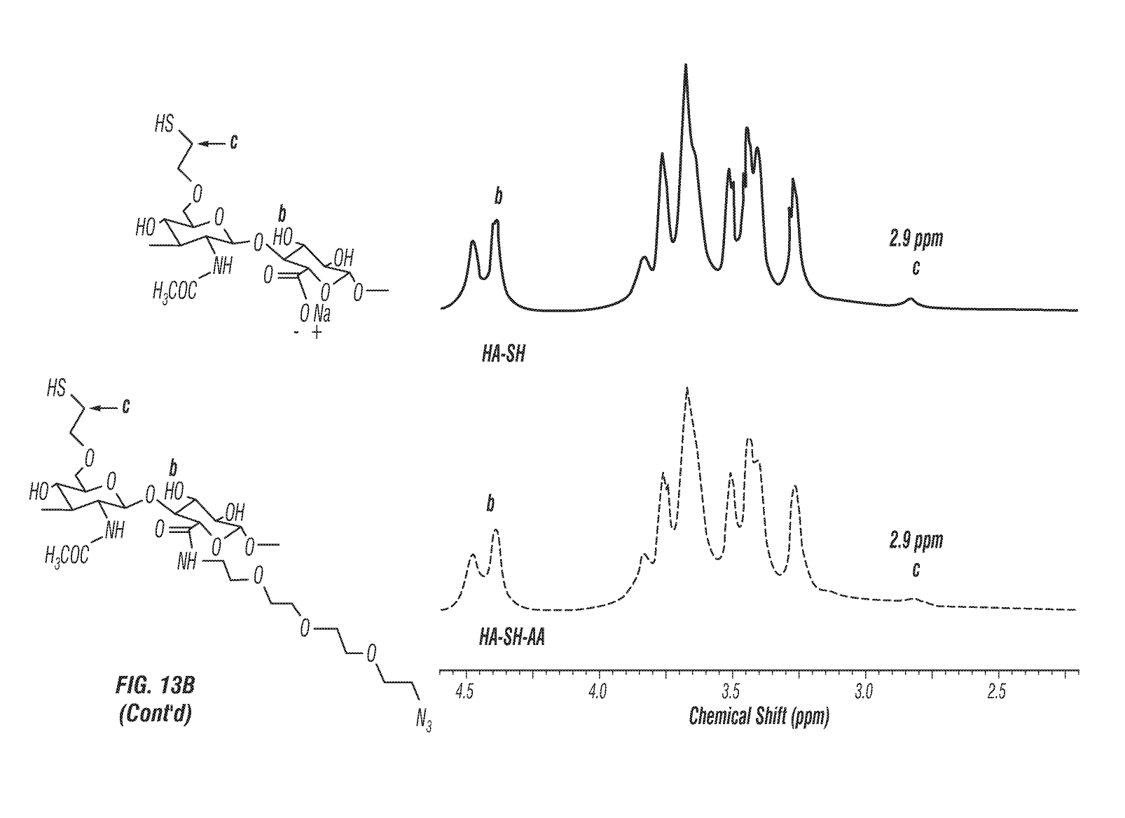

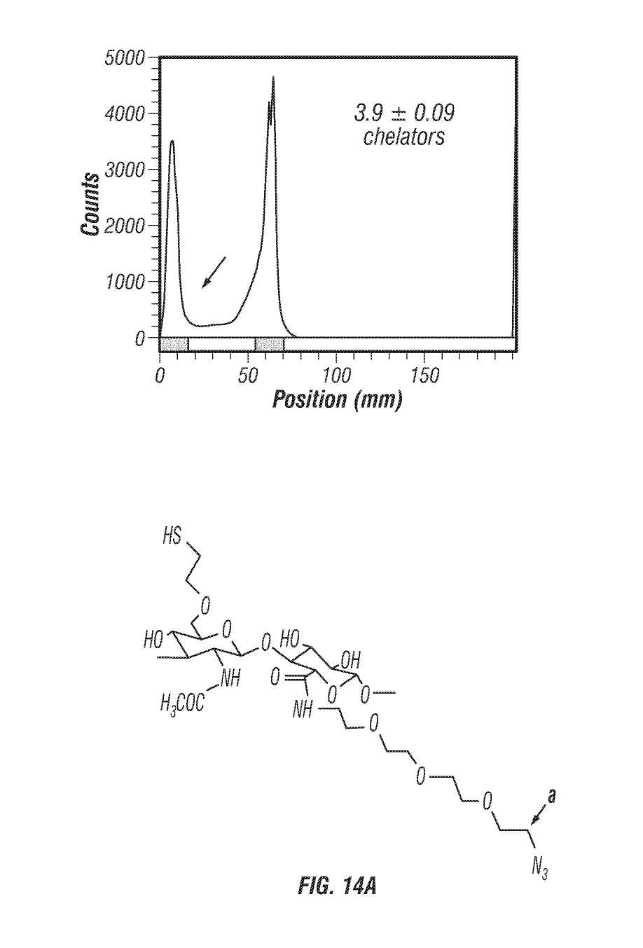

The preliminary characterization of thiol group functional HA backbone is shown in FIGS. 13A-B. The 2.8 ppm peak on the ¹H NMR spectrum and positive response to the TNBS assay were only present on HA samples functionalized with thiol, or thiol and azide groups. (n=3, p*<0.05). The degree of thiolation determined by ¹H-NMR was 12.44±1.06% on HASH and 8.78±0.68% on dif HA.

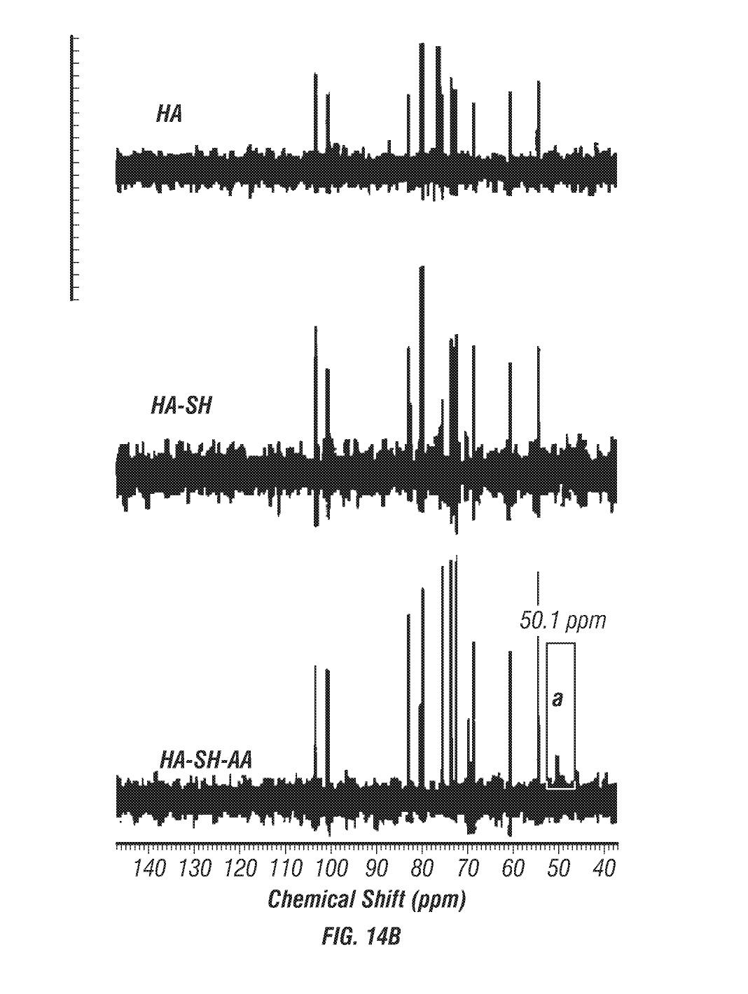

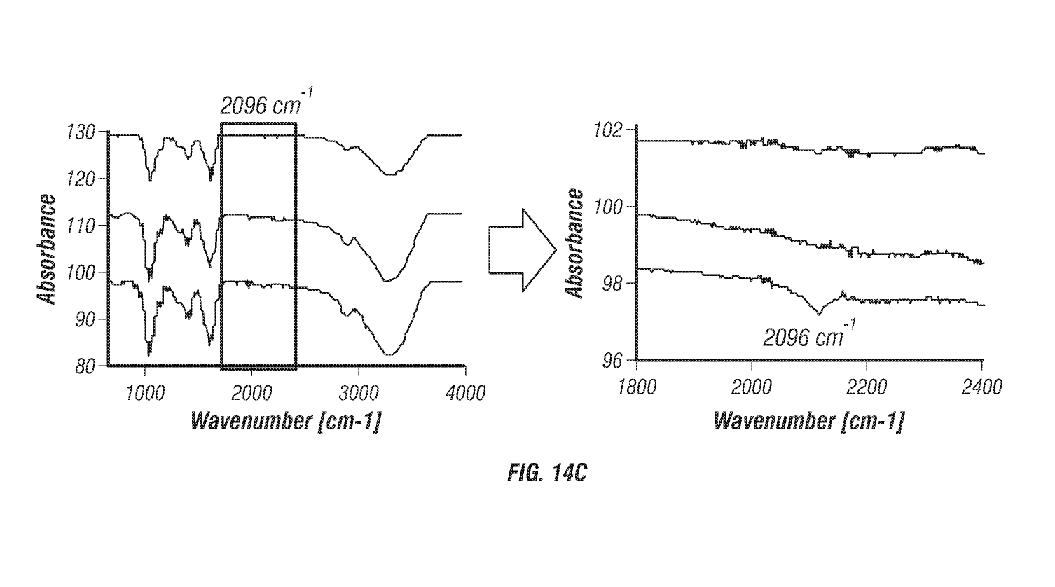

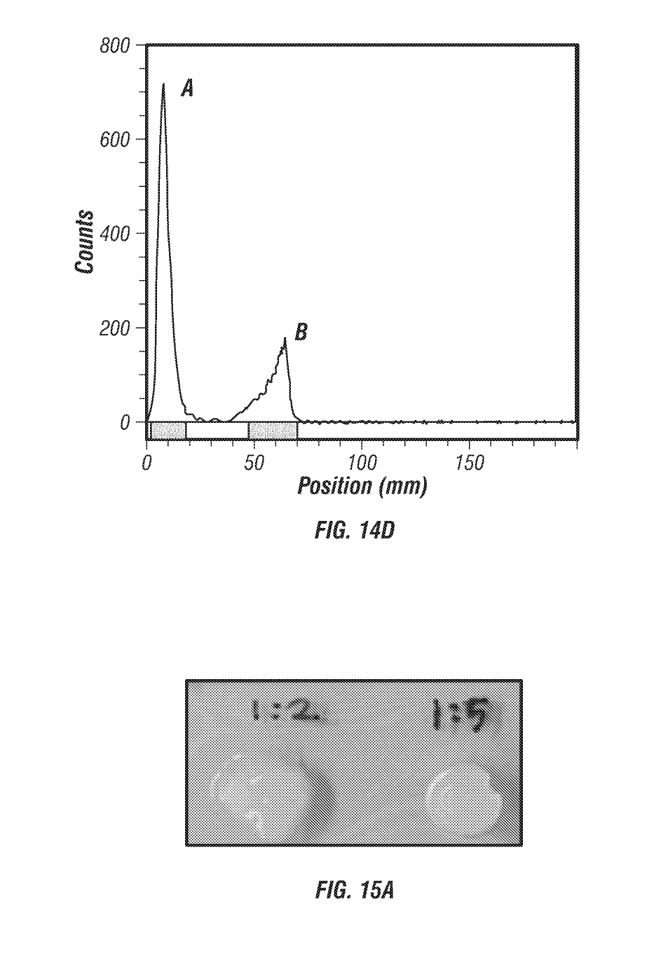

Characterization of azide group on HA is shown in FIGS. 14A-C. The azide group peak was presented at 2096 cm⁻¹ on the FT-IR spectrum, and at 50.1 ppm on the ¹³C-NMR spectrum. These peaks are not present in the HA and HASH spectrums. Previous reports of azide grafted HA have not quantified the amount of functionalization on the backbone (Crescenzi, et al., 2007; Takahashi, et al., 2013). An approach was adopted that is well established in the field of radiolabeled antibodies (Ghosh, et al., 2013; Meares, et al., 1984) to monitor the reaction of DOTA-DBCO with the azide-functionalized HA. Introduction of the chelating agent permitted radiolabeling with the positron-emitting radiometal ⁶⁴Cu, and radio-TLC analysis of the sample quantified the azide content grafted to HA. The ratio of peaks representing labelled product and unreacted ⁶⁴Cu was used to calculate a chelator/HA ratio of 8.9±0.07 (FIG. 14D), suggesting the presence of nearly 9 azide groups per HA, which is approximately a 4% functionalization of HA. Recent reports (Sun, et al., 2015; Hoogenboom, 2010; Hensarling, et al., 2009; Fairbanks, et al., 2010) suggest the possibility of a thiol-yne side reaction between the alkyne and two sulfhydryl groups in the presences of a radical source, such as UV light. Although the quantification reaction was not knowingly exposed to a radical source, it is possible that azide functionalization may be less than 4% due to a thiol-yne side reaction with DBCO. While it is possible to increase the amount of azide functionalization, the low azide functionalization may be sufficient for tethering the bioactive molecules to promote neural differentiation as the suggested optimal peptide concentration to promote neural differentiation of mES in 3D culture is low (Yang, et al., 2015). The average number of chelating moieties capping the azide group in HA was 3.9±0.09 on radio TLC on diHA samples.

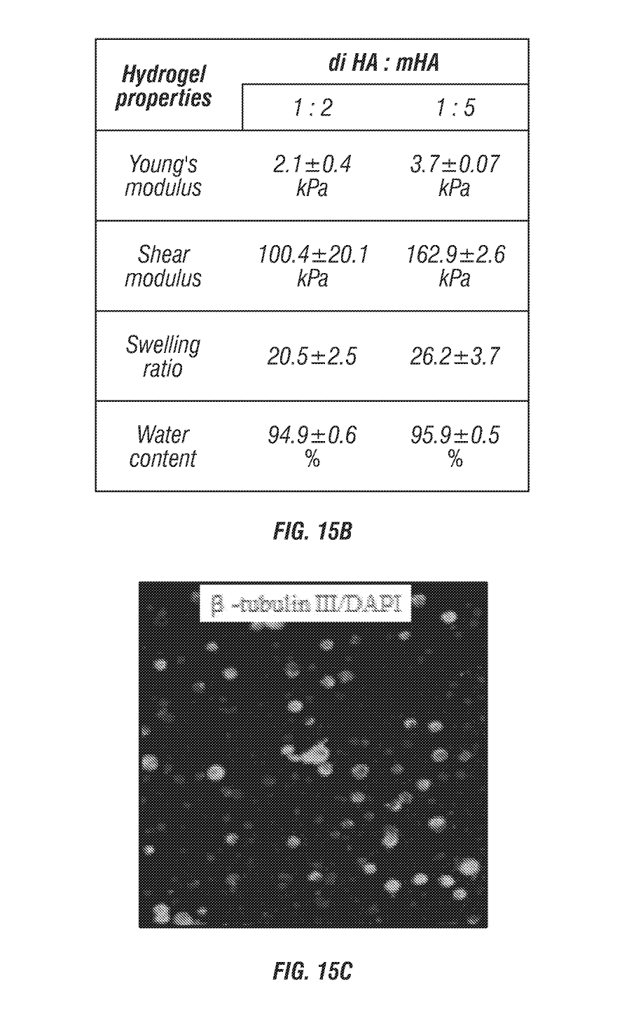

Characterization of diHA/mHA hydrogels of various concentrations is shown in FIGS. 15A-C. A higher ratio of mHA increased the Young's modulus and shear modulus. Neural differentiation of mouse embryo stem cells (mES) were examined in 3D HA hydrogels. 44.51±0.4% of mES expressed β-tubulin III on day 3 of differentiation in 1:2 mixing ratio HA hydrogel. (n=5, p*<0.05, p***<0.001). Increased overall molecular rigidity and decreased overall molecular movement could explain the increased Young's Modulus and swelling ratio observed in the 1:5 hydrogel compared to the 1:2 hydrogel. As the ratio of thiol to acrylate changes between formulations, so does the potential for step-growth polymerization caused by either Michael addition, thiolene chemistry, (Mergy, et al., 2012; Shih and Lin, 2012) and oxidation induced crosslinking (Tingaut, et al., 2011).

Step-growth polymerization in hydrogels can lead to higher mechanical properties then observed in chain growth polymerized hydrogels (Lin, et al., 2011). Increased step-growth polymerization in the 1:5 hydrogel compared to the 1:2 hydrogel could be contribution to the increase in observed Young's Modulus. As the 1:2 hydrogel has a higher thiol content, it is more likely to form oxidation induced disulfide bonds than the 1:5 hydrogel. The formation of these disulfide bonds during polymerization would reduce the concentration of total reactive moieties and skew covalent polymerization toward chain growth, both of which would lead to reduced mechanical properties. Oxidation induced crosslinking due to disulfide bond formation has been linked to a reduced swelling ratio in hydrogels (Shu, et al., 2002), which is consistent with the observed data (Table 6).

TABLE 6
Material properties of di-functional hyaluronic acid (dif HA)/
methacrylate hyaluronic acid (mHA) hydrogel of various mixing ratios.

dif HA:mHA

| Properties | 1:2 | 1:5 |
|---|---|---|
| Young's modulus [kPa] | 2.1 ± 0.4 | 3.7 ± 0.07 |
| Swelling ratio | 20.5 ± 2.5 | 26.2 ± 3.7 |
| Water content [%] | 94.9 ± 0.6 | 95.9 ± 0.5 |

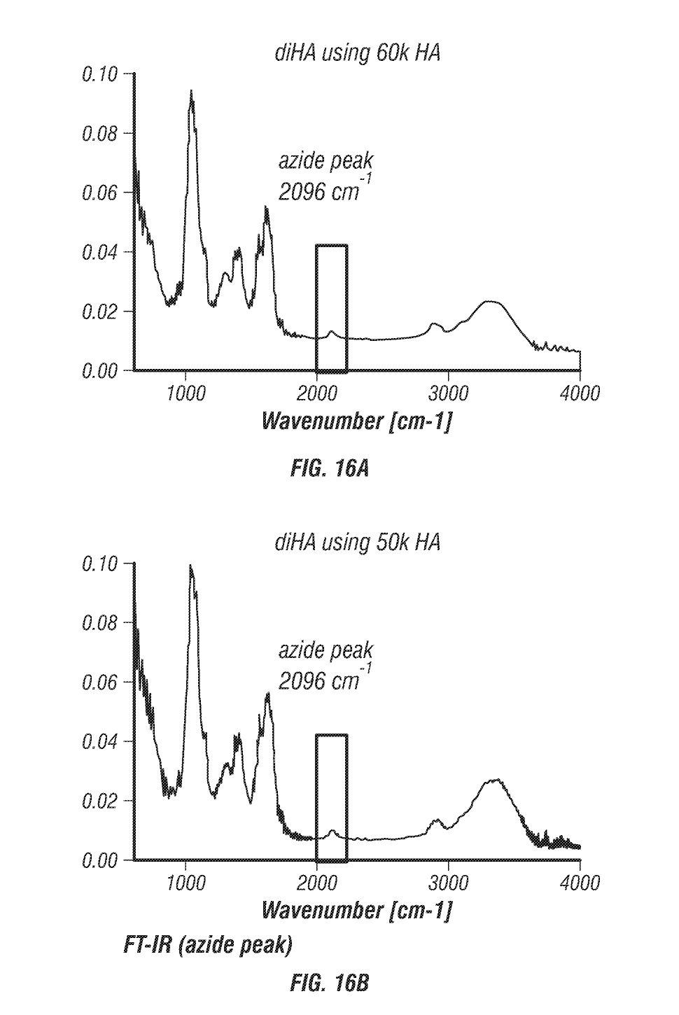

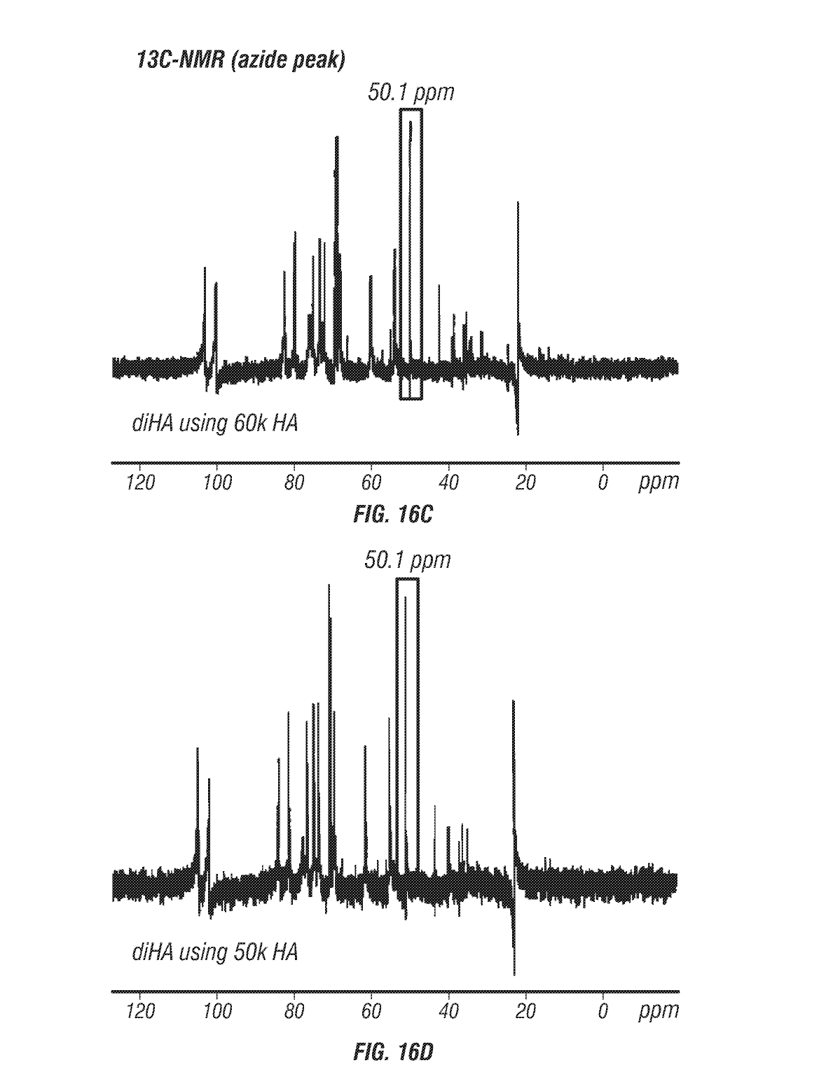

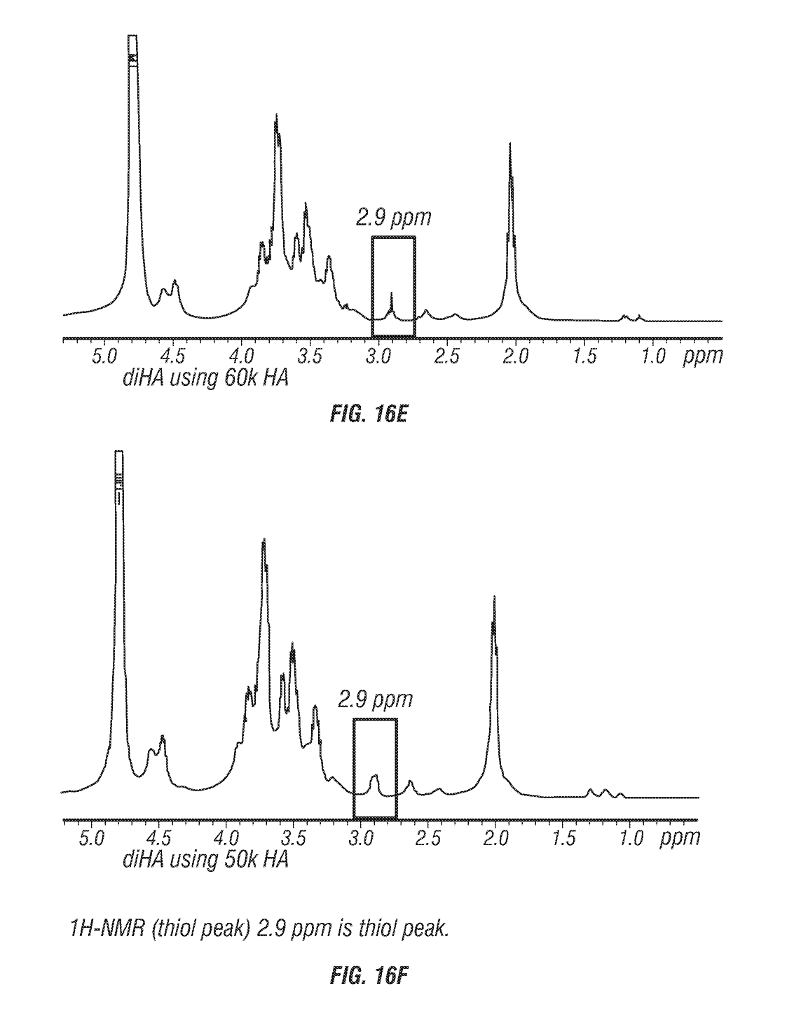

Characterization of diHA hydrogels synthesized using different molecular weight HA for the process is shown in FIGS. 16A-F. For diHA synthesized using 50,000 mw HA (PEGWORK) the result from FT-IR analysis (azide peak identified) on diHA using 50,000 mw HA is shown in FIG. 16B, 13C-NMR analysis of this diHA (azide peak identified) is shown in FIG. 16D, and 1H-NMR analysis of diHA to identify the thiol peak (which was 20%) is shown in FIG. 16F. Characterization of diHA hydrogels synthesized using 60,000 mw HA (PEGWORK) the result from FT-IR analysis (azide peak identified) is shown in FIG. 16A, 13C-NMR analysis of this diHA (azide peak identified) is shown in FIG. 16C, and 1H-NMR analysis of diHA to identify the thiol peak (which was 16%) is shown in FIG. 16E.

A number of factors may contribute to the altered degradation rates observed between tested hydrogel formulations (FIG. 3). Increases in HA functionalization have been shown to slow hydrogel degradation (Chung, et al., 2006), while changes in the moiety functionalize on the backbone or the ratio of moieties on the backbone have been shown to alter HA hydrogel degradation rates (Schantd, et al., 2012; Vanderhooft, et al., 2009). Due to the number of factors that can affect degradation rate beyond those previously discussed (molecular weight of HA, inclusion of non-HA crosslinkers, etc.), direct comparisons between previous published studies is difficult. However, previous studies were found in the literature that used a similar molecular weight of HA and degradation protocol. The complete degradation of 2% mHA hydrogels (12% functionalization) within 24 hrs (Burdick, et al., 2005; Chung, et al., 2006) and complete degradation of 1% HASH (7.5% functionalization) mixed with 2-dithiopyridyl HA (5.5% functionalization) hydrogels in 4 days (Zhang, et al., 2016). The tested hydrogel formulations tested persisted for at least an additional 24 hrs than the previous studies' hydrogel formations before complete degradation. See FIG. 21. This increased durability may not be particularly critical for biologic applications.

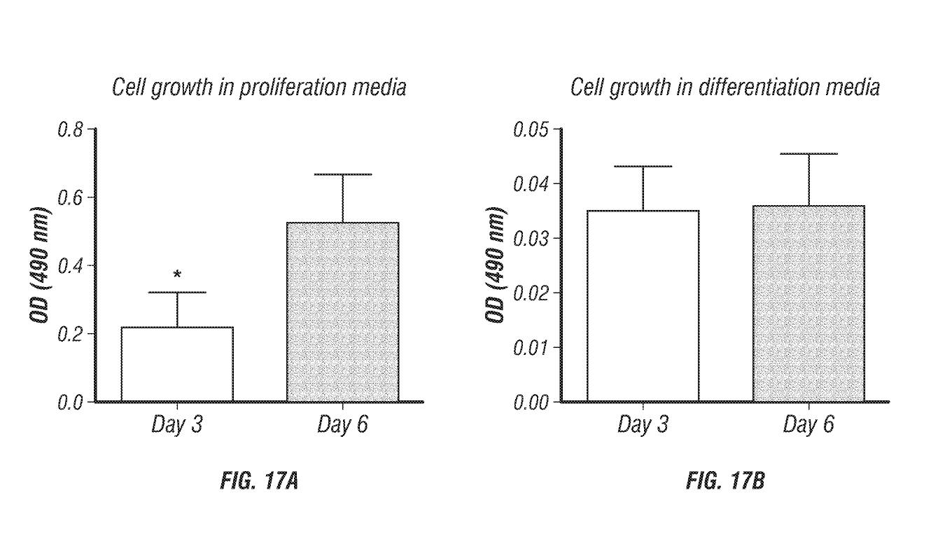

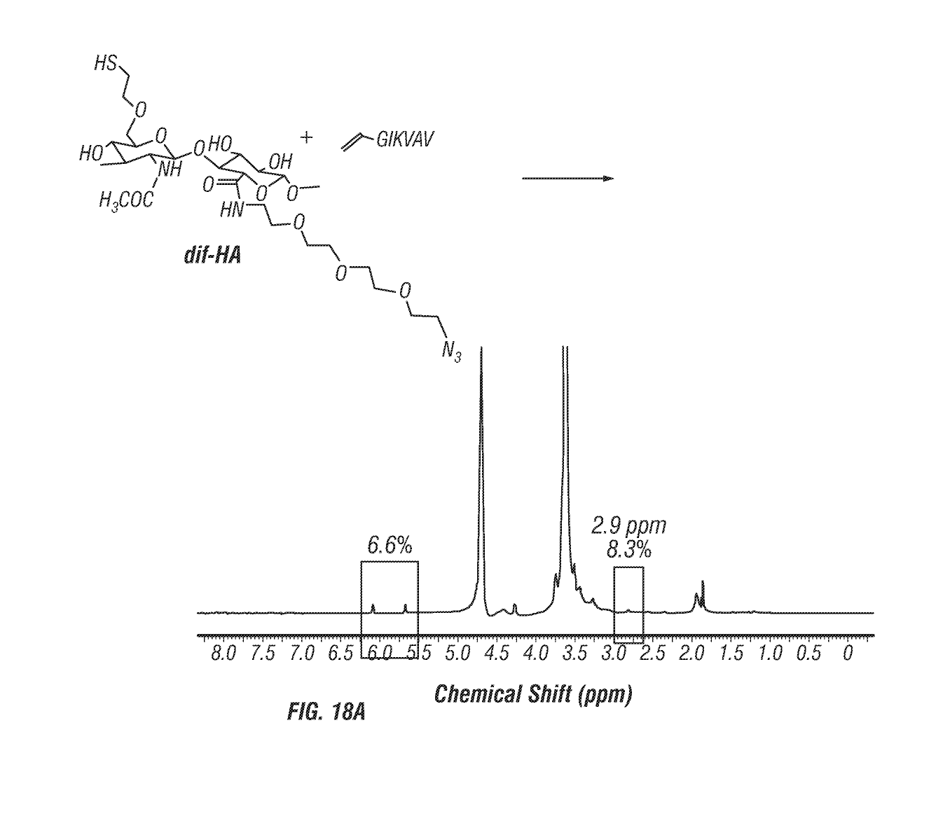

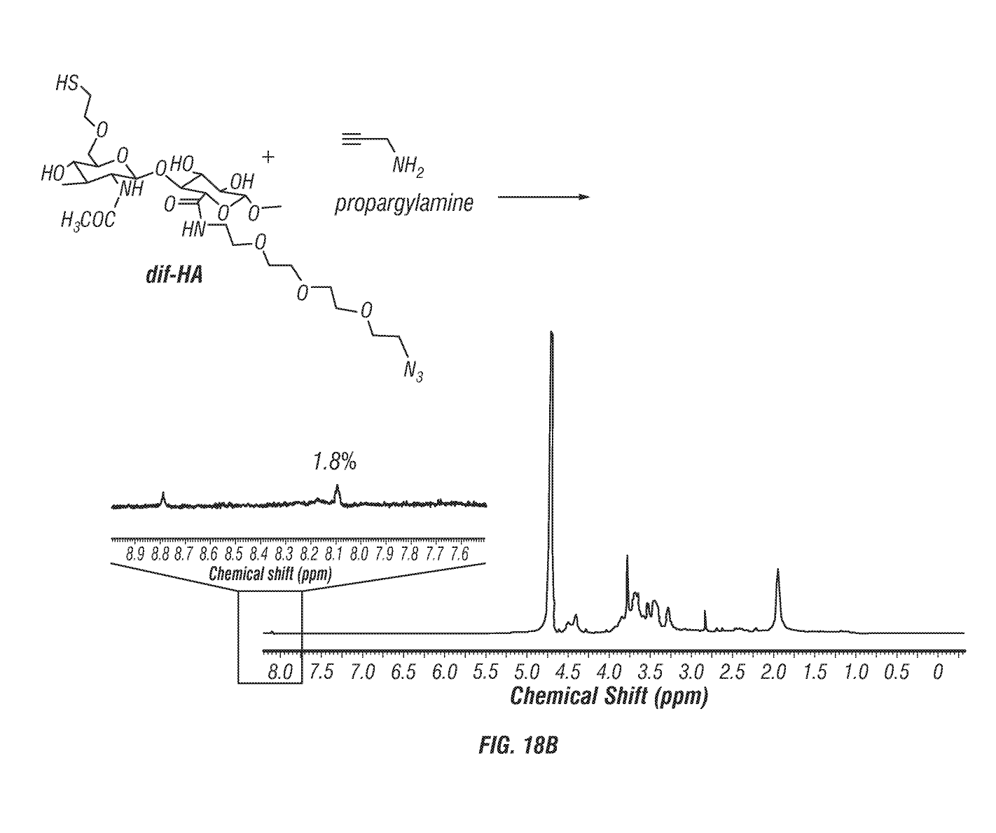

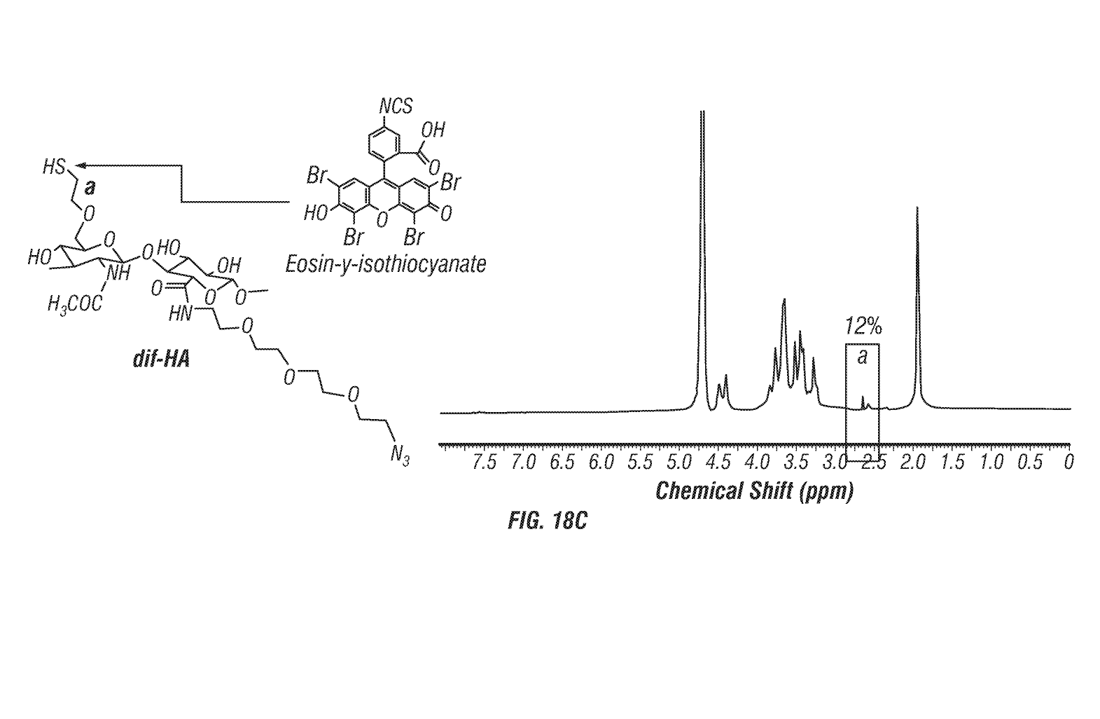

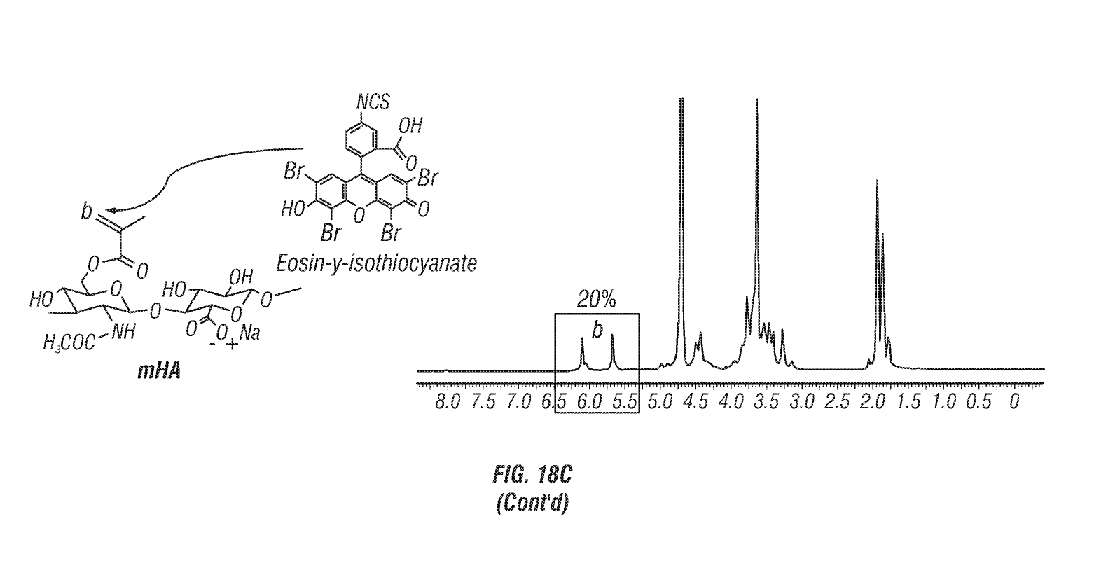

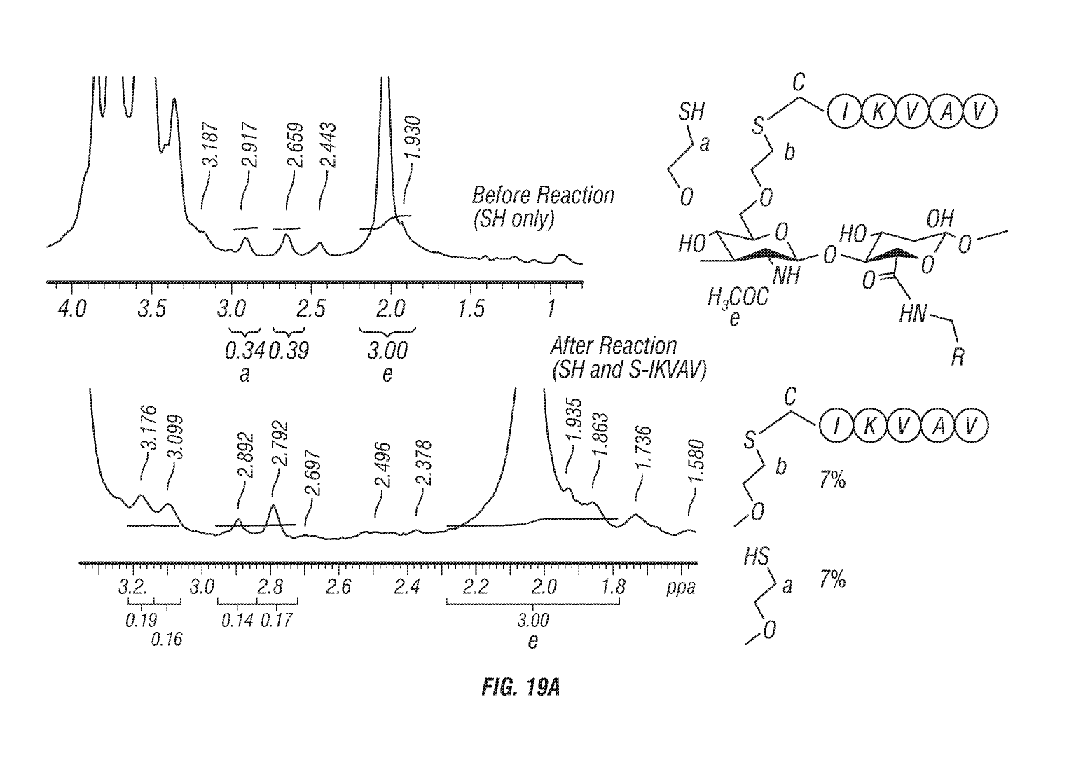

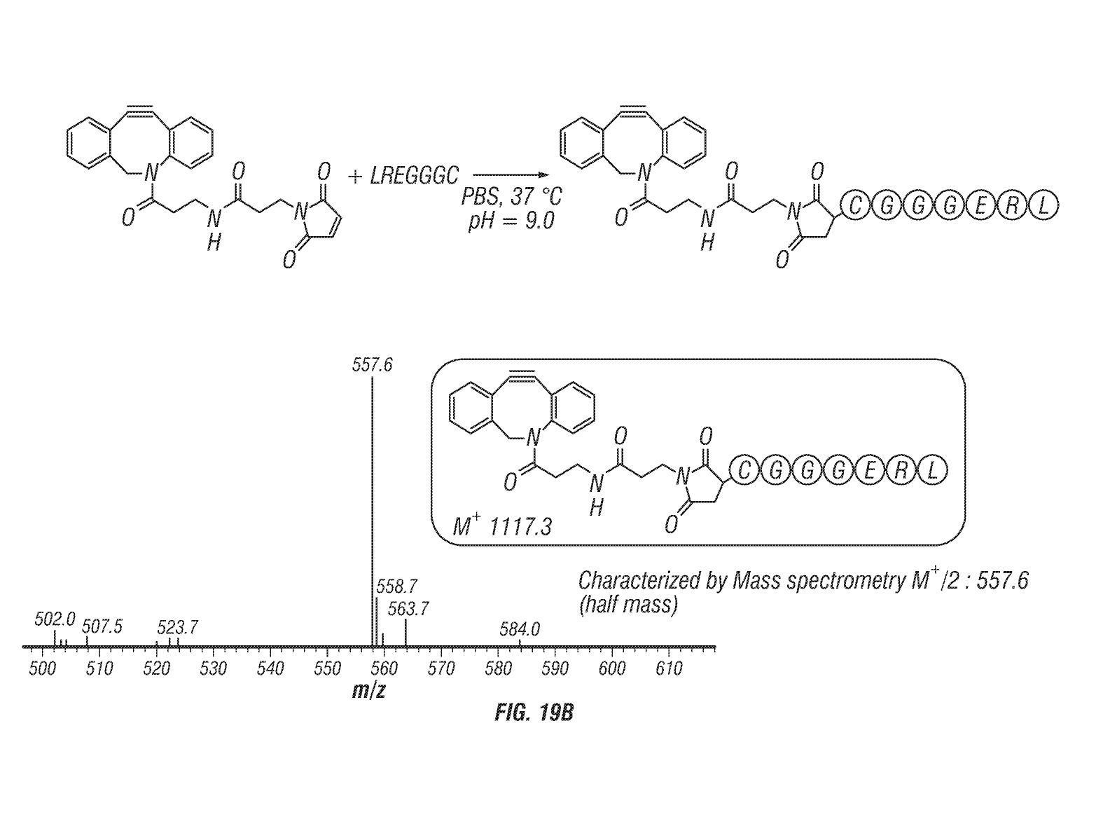

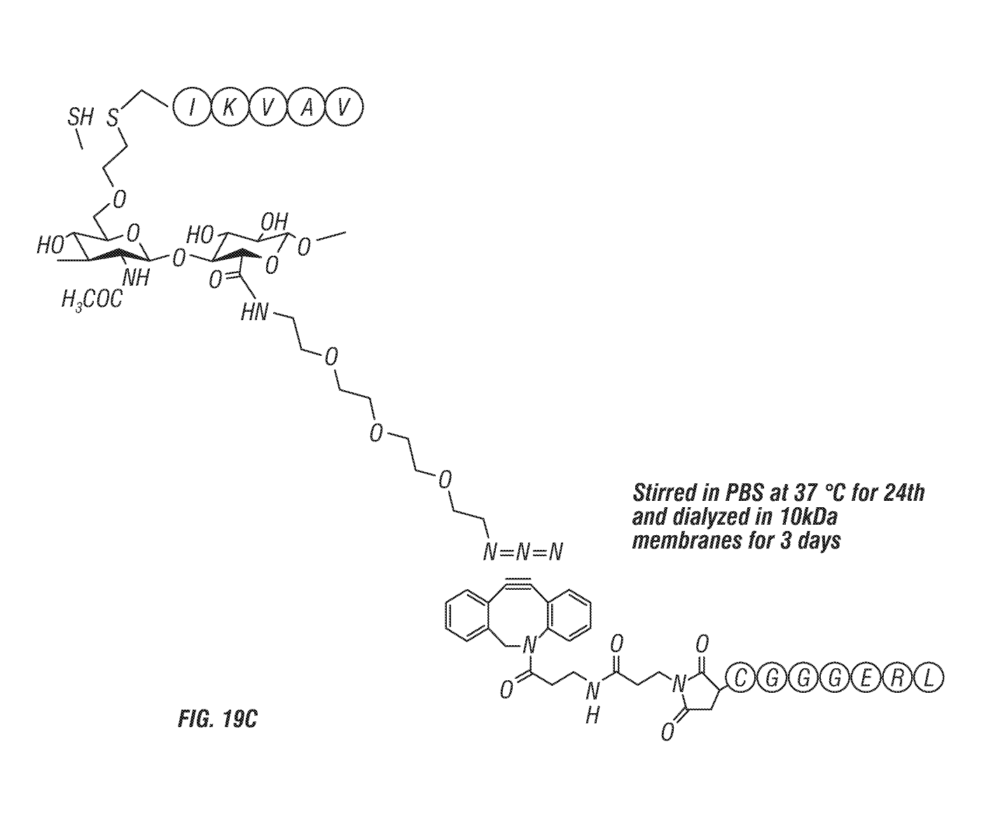

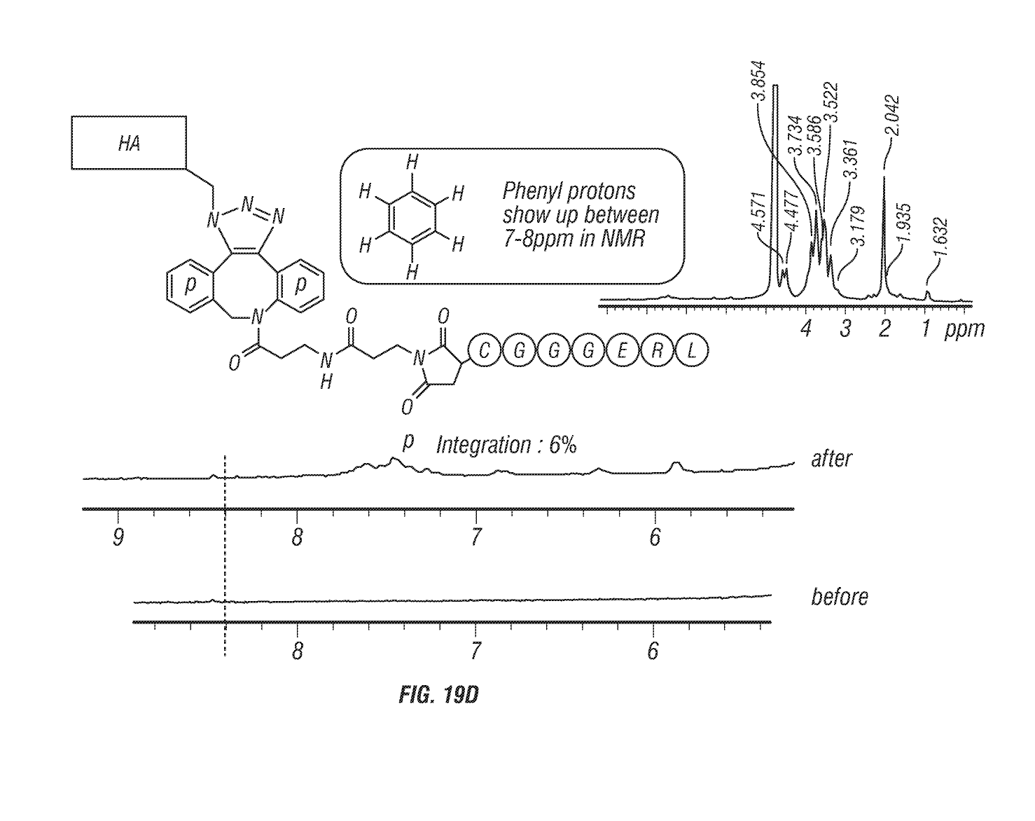

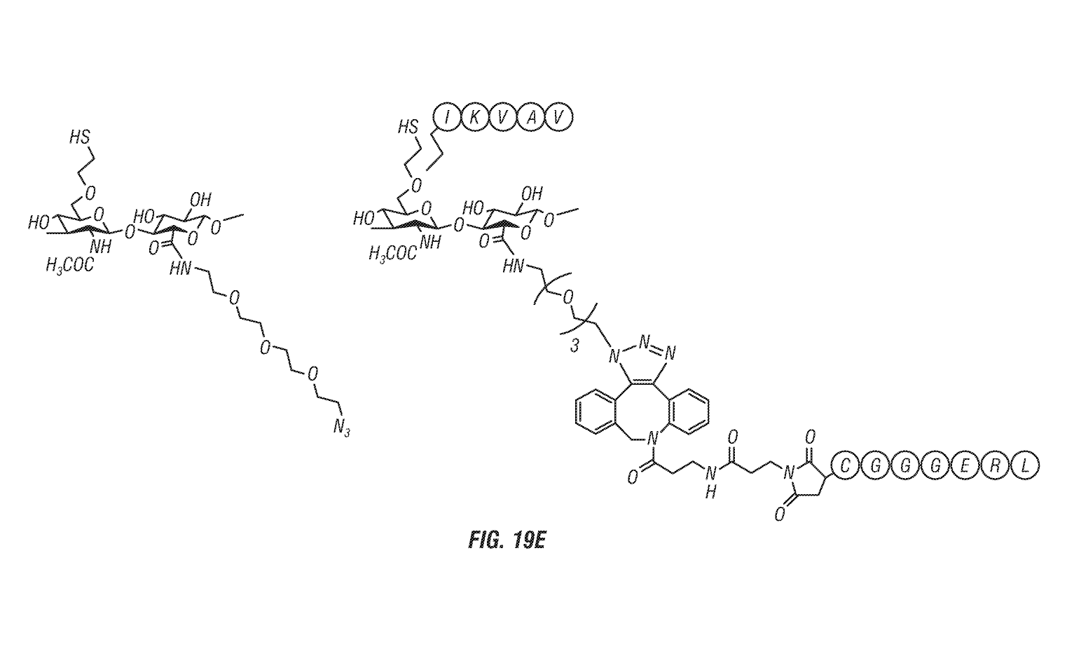

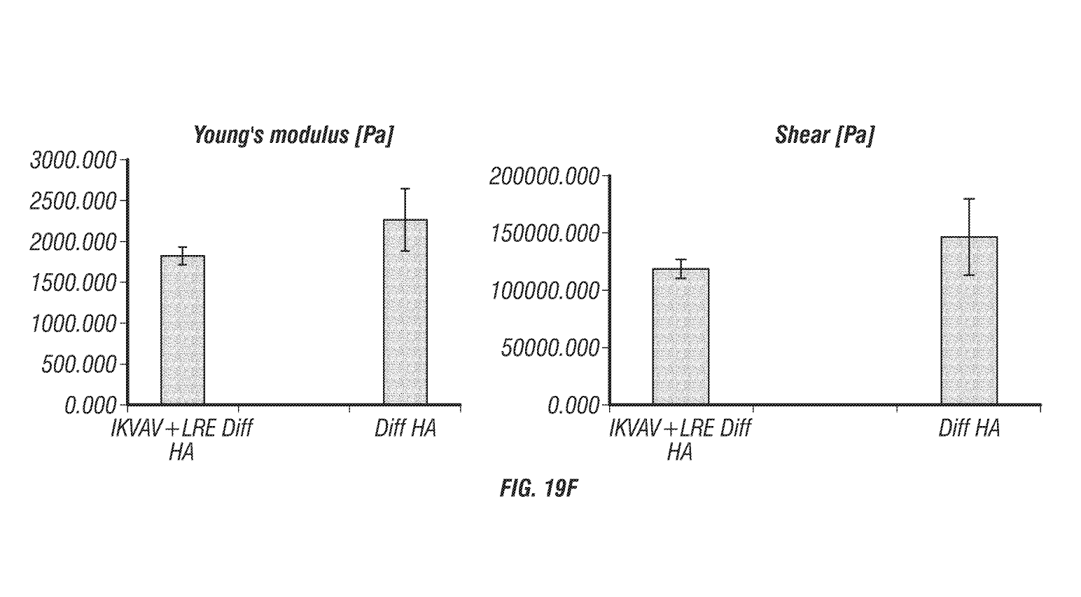

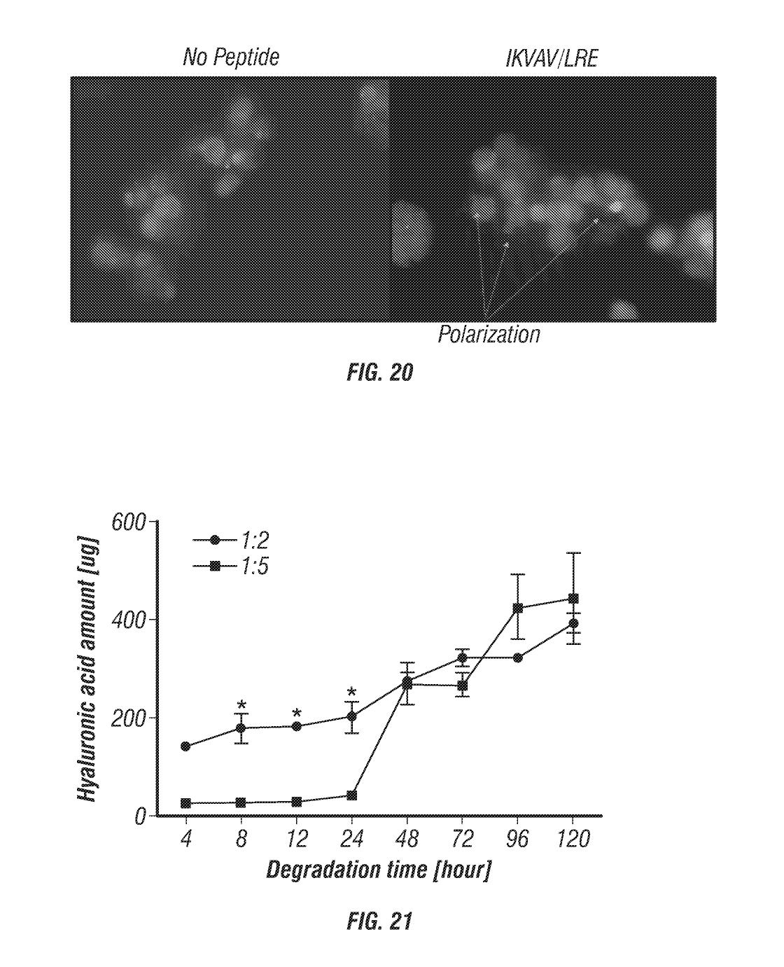

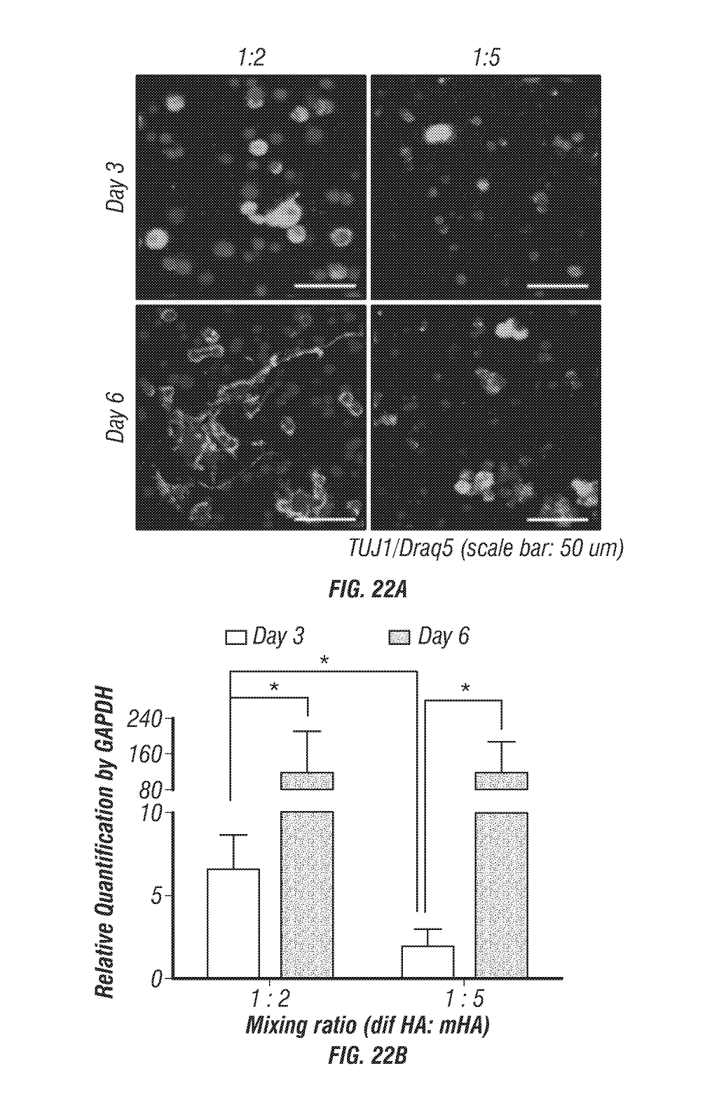

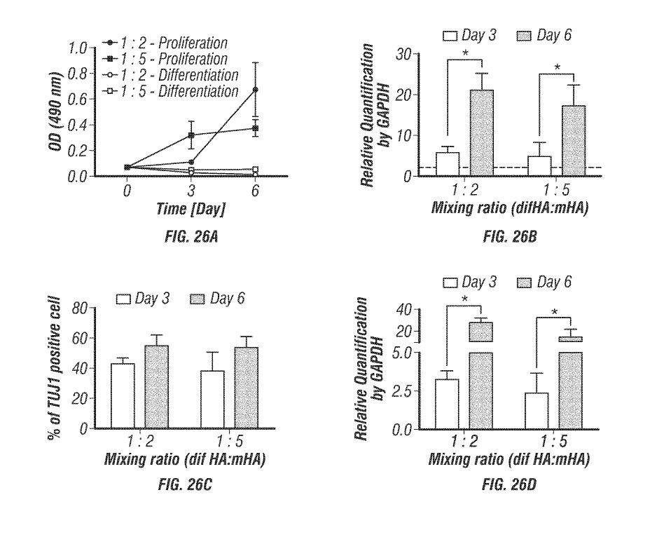

A time course of mouse embryonic stem cell growth over time under growth conditions in proliferation and differentiation media (FIGS. 17A-B) indicated that the cells maintain a consistent cell number as they differentiate on diHA and multiply when placed in growth conditions. Preliminary findings indicate that human cells can also attach and grow on a diHA matrix. These results indicate that thiol and azide functional groups were presented on the modified and crosslinked HA hydrogel, simultaneously, mES can attach and differentiate on base matrix without additional bioactive signaling. Modified HA with two independent linkers facilitating the attachment of two independent bioactive signaling molecules may be useful tools to mimic native ECM facilitating the restoration of neurological functions and allow independent control of the amount of each bioactive molecule present. Modification of the HA backbone is known to effect HA bioactivity (Collins and Birkinshaw, 2013). mES were encapsulated in 3D HA hydrogels for 6 days to access the effects of dif HA containing hydrogels on cellular proliferation and neural differentiation. The growth curve for mES in encapsulated in 1:2 and 1:5 (dif HA:mHA) formulations showed a similar trend between formulations in both pluripotent proliferation and neural differentiation media over the time course (FIG. 21). However, a reduction in doubling time was observed for mES encapsulated in the 1:2 hydrogels (45.26 hr) compared to mES encapsulated in 1:5 hydrogels (61.31 hr) cultured in pluripotent proliferation media. Over the time course, significant increases in the mRNA expression of immature neuronal markers, PAX6 and TUJ1, and the percentage of cells expressing TUJ1 protein were observed in mES encapsulated in both formulations, but were not significant between formulations (FIG. 26). However, cellular polarization was visible on day 3, and long neurite extension was visible on day 6 in cells encapsulated in the 1:2 hydrogel, but not cells encapsulated in the 1:5 hydrogel (FIG. 22A). At day 3, mRNA expression of MAP2, a mature neuronal marker, was expressed at a significantly higher level in cells encapsulated in the 1:2 hydrogel compared to cells encapsulated in the 1:5 hydrogel (FIG. 22B). Both the differences in neurite extension and maturation between formulations were unexpected as typically the addition of bioactive signaling molecules are necessary to promote cellular adhesion and differentiation in hydrogels fabricated from modified HA (Wang, et al., 2012; Hou, et al., 2005).

Several factors potentially contribute to the observed difference in cellular response between formulations. The increased early degradation in the 1:2 hydrogel compared to the 1:5 hydrogel (FIG. 21) could be promoting the observed axon extension by reducing the number of obstacles impeding neurite extension in the hydrogel (McKinnon, et al., 2013). Typically, increases in backbone functionalization has a negative impact on cell behavior (Bencherif, et al., 2008; Carmi-Unal, et al., 2013). Changes in the type and degree of modification to the HA backbone affect chain mobility (Ambrosio, et al., 1999), which could affect cellular binding to HA (Banerji, et al., 2007). Multiple stimuli, including the media supplement retinoic acid, can cause MAP2 expression during neural differentiation and lead to different expression profiles of the protein in the cells (Fischer, et al., 1986). Without wishing to be bound by any theory, it is believed that the increased degradation allows more retinoic acid to reach the mES in the 1:2 hydrogel than in the 1:5 hydrogel or that greater great cellular binding to HA by the mES in the 1:2 hydrogel than in the 1:5 hydrogel is accelerating the neural differentiation. As MAP2 expression is precursor to neurite extension (Dinsmore and Solomon, 1991), the higher early MAP2 expression in mES cultured in the 1:2 hydrogel compared to the 1:5 hydrogel likely contributes to the observed differences in neurite extension between the formulation.

Although the Young's Moduli for both formulations (Table 2) falls within the range reported for brain tissue (Moore and Sheetz, 2011), neural stems cells can be sensitive to alterations in mechanical properties of their substrate (Leipzig and Shoichet, 2009), causing changes in cellular attachment and neurite extension (Balgude, et al., 2001; Jiang, et al., 2008). Conflicting reports exist in the literature regarding the sensitivity of mES during neural differentiation to changes in Young's Modulus (McKinnon, et al., 2013: Ali, et al., 2015; Norman and Aranda-Espinoza, 2010). Neurite extension and mRNA gene expression of neural differentiation markers by embryonic neurons and mES differentiating toward the neuronal linage have been reported to be unaffected by changes in the Young's modulus of the substrate in two-dimensional (2D) culture (Ali, et al., 2015; Norman and Aranda-Espinoza, 2010), while another study found reduced neurite extension of with increased Young's Modulus in three-dimensional (3D) culture (McKinnon, et al., 2013). However, the 3D study used increases in the polymer mass fraction to increase the Young's Modulus, which would change additional material properties in the hydrogel that could have hindered neurite extension and complicates the assessment that the modulus change alone was the cause of the observed biological results (Smith Callahan, et al., 2013). The current study used a consistent polymer mass fraction between formulations that reduces the number of material properties that could change between formulations compared to the previous 3D study. The contribution of the Young's modulus change to the observed biological results remains unclear due to alterations in additional material properties that would have greater effect on the biological results in the present 3D study than the previous 2D studies (Smith Callahan, 2016).

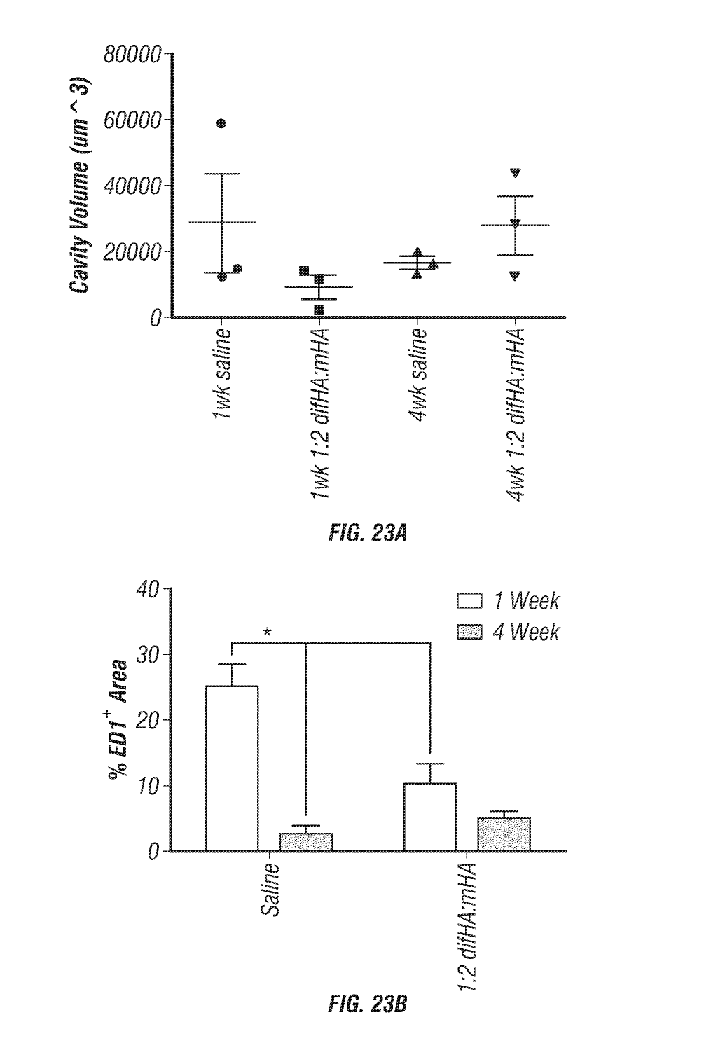

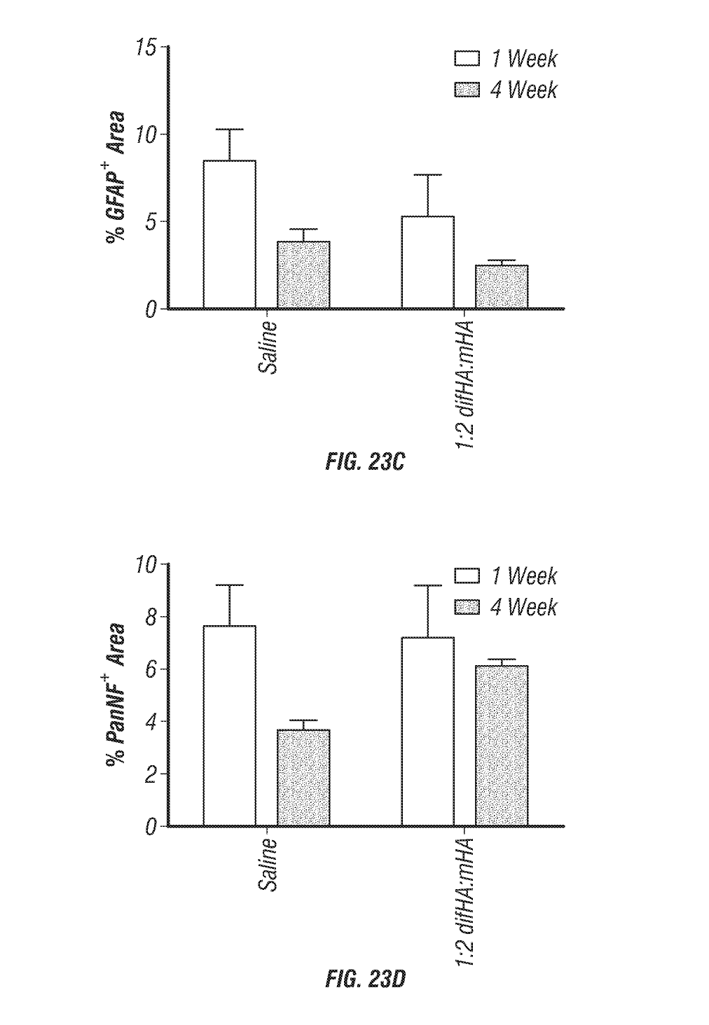

Based on in vitro results, the 1:2 difHA:mHA formulation was selected for preliminary testing in a contusion model of rodent spinal cord injury. Compared to saline injection, the 1:2 hydrogel was found to significantly reduce ED1⁺ macrophages in the injured spinal cords 1 week after implantation (FIG. 23B), but did not significantly influence the cavity volume or the area of GFAP⁺ or pan-axonal neurofilament⁺ staining within the injured spinal cords over the examined time course (FIG. 23).

The in vivo gelation relied solely on Michael addition for covalent bond formation. It is possible that not all the thiols were consumed during Michael addition (8; Shu, et al., 2004). Conflicting reports in the literature regarding whether HASH or other materials containing free thiols cause an inflammatory response (Zheng, et al., 2004, Khaing, et al., 2011; Lam, et al., 2014; Young, et al., 2013). Previous studies of HA matrices that did not contain a thiol have found ED1 expression to either not differ substantially from control (Mothe, et al., 2013) or to be reduced to control (Gupta, et al., 2006). The current study found ED1 expression in matrix injected spinal cords to be significantly reduce at 1 week compared to control, but similar to control after 4 weeks of implantation, indicating that the 1:2 dif HA:mHA matrix did not evoke a significant inflammatory response at the time points studied.

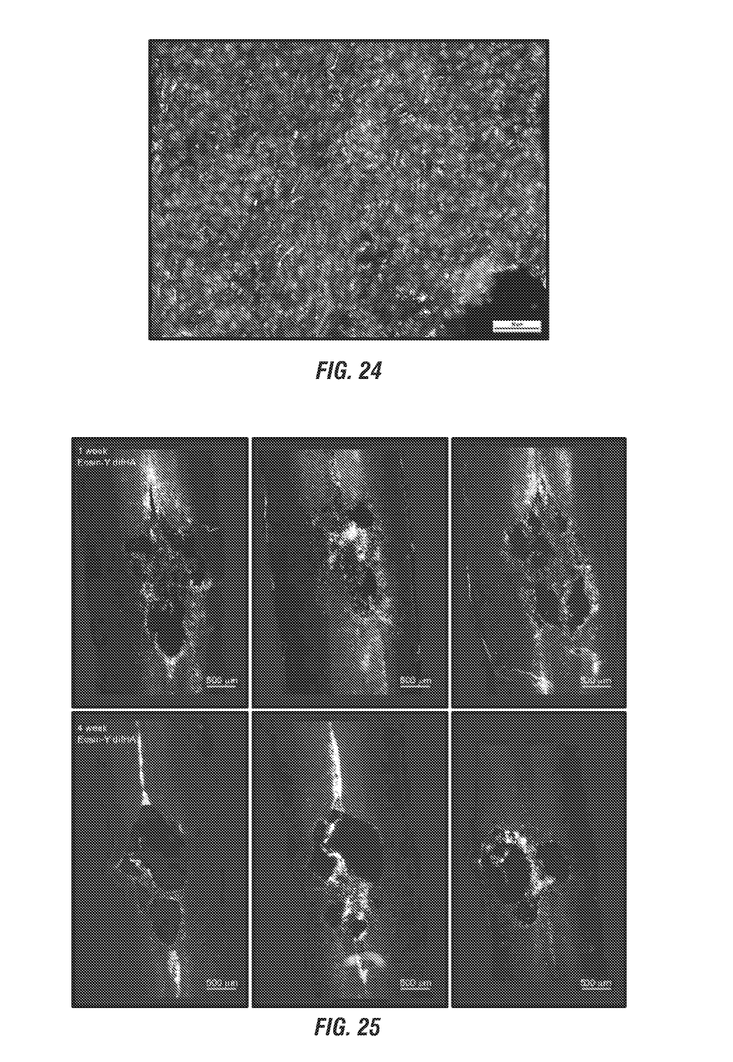

Typically, inclusion of a HA matrix leads to reduction of lesion volume (Mothe, et al., 2013; Gupta, et al., 2006; Austin, et al., 2012). Increases in axons post injury with HA matrices compared to control have, also, been observed (Austin, et al., 2012). However, neither was observed in the present study. However, axons were found to interact with the eosin-tagged HA matrix at the 1 week time point (FIG. 24), indicating the matrix is supportive of invasion host axon invasion without additional bioactive peptide signaling. The addition of bioactive signaling should lead to further enhancements of this invasion and potentially increases in tissue sparing compared to control. Differences in experimental design between the previous studies and the current study, such as the type of spinal cord injury, the time between injury and matrix implantation and the inclusion of additional bioactive signaling molecules, likely have significant effects on the observed results. Many studies of matrices in spinal cord injury implant the matrix immediately after injury (Zheng, et al., 2004, Khaing, et al., 2011; Gupta, et al., 2006; Horn, et al, 2007; Kushchayev, et al., 2016). This is clinically impractical as most with patients with spinal cord injury have multiple injuries and must be stabilized before any treatment to improve long term neurological function could be performed (Rubinos and Ruland, 2016; Liu, et al., 2016; Witiw and Fehlings, et al., 2015), which is why the injection of the matrix was delayed two weeks in the current study.

In order for the matrix to be beneficial crosslinking must occur (Liang, et al., 2013). It is possible that Michael's addition did not sufficiently occur and oxidation induced crosslinking established the initial gel structure in vivo (Tingaut, et al., 2011). These oxidative induced disulfide bonds would be susceptible to changes in pH (Shu, et al., 2002) and water absorption (Choh, et al., 2011). As the lesion environment is known to change over time (von Leden, et al., 2016; Volpato, et al., 2013), the lesion environment could become unsupportive to the maintenance of disulfide bonds, which would speed the clearance of the matrix from the area and limit its effects on tissue regeneration (FIG. 25). Several studies suggest that HA matrices can be maintained in the spinal cord lesion for at least 2 months (Khaing, et al., 2011; Kushchayev, et al). However, one study monitoring in vivo degradation found the tested HA scaffold to completely degrade within 3 days (Zhang, et al., 2016). It is unclear in the present study, if failure to form covalent bonds, or enzymatic degradation of the matrix led to the observed loss of matrix structure in the lesion at 4 weeks. To address this issue future studies will include a free radical initiator to facilitate covalent bond formation.

### Example 9—Methods and Materials

Materials and Analytical

HA was purchased from Lifecore (Chaska, Minn.). ¹H and ¹³C NMR spectra (Bruker, Billerica, Mass.) were recorded at 600, or 150 MHz in D₂O and were referenced to the residual proton. The degree of functionalization was probed by NMR analysis upon normalization to the integrals of the HA backbone methyl peak. Fourier Transform Infrared Spectroscopy (FT-IR) spectra were recorded on Nicolet iS10 with Attenuated Total Reflectance system (Thermo Fisher Scientific, Waltham, Mass.) using powder samples, and scanned 32 times from 600 cm⁻¹ to 4000 cm⁻¹, with a 2 cm⁻¹ resolution. Radiolabeled samples were analysed by radio-thin-layer chromatography with an AR-2000 scanner (Eckert & Ziegler Radiopharma, Hopkinton, Mass.). Chemicals were purchased from Thermo Fisher Scientific (Waltham, Mass.) or VWR International (Radnor, Pa.) unless specified otherwise.

Synthesis of Methacrylate HA (mHA)

HA was modified with methacrylate groups in a manner similar to previously described (FIG. 6) (Smeds, et al., 2001). Briefly, 1.0% (w/v) solution of sodium hyaluronic acid in DDW was slowly mixed with a 10-fold molar excess of methacrylic anhydride (Sigma-Aldrich, St. Louis, Mo.) at 4° C. overnight. The pH was maintained between 8 and 9 using 10 N NaOH. The methacrylated HA (mHA) was precipitated with a 10-fold volume of chilled 95% ethanol, and pelleted with centrifugation at 2,000 rpm for 10 min. The supernatant was removed, the pelleted mHA was dissolved in DDW, and then dialyzed (MWCO=8-10 kDa) against DDW for 2 days with DDW changed several times per day. The solution was frozen at −20 OC overnight, lyophilized to obtain mHA powder, and analyzed by ¹H-NMR (Supporting information Figure S1b). Based on ¹H-NMR, approximately 33% of the available targets on the HA backbone were functionalized.

Colorimetric Quantification of Thiol Content

The thiol concentration within the HASH and dif HA products was determined in a manner similar to previously described using Ellman's Method (Serban, et al., 2008). Briefly, HA, HASH, or dif HA was dissolved in 5,5′-Dithiobis-2-nitrobenzoic acid (DTNB, 2 mg/mL in PBS) and the resultant solution was stirred overnight at room temperature. The DTNB-HA, -HASH and -dif HA solution were dialyzed (MWCO=8-10 kDa) against DDW for 3 days with DDW changed daily, frozen overnight at −80° C., and then lyophilized. DTNB-HA, -HASH and -dif HA were dissolved in PBS (2 mg/mL), and 1% (w/v) DTT was added. The absorbance at 412 nm was read using a microplate reader (Tecan Infinite M1000, Maennedorf, Switzerland). The value of DTNB-HA derivatives was normalized to unmodified HA as a control.

Quantification of Azide Content by Radio-Thin Layer Chromatography

A modified dibenzocyclooctyne (DBCO) analog of the widely used bifunctional chelating agent, 1,4,7,10-tetraazacyclododecane-N,N′,N″,N″′-tetraacetic acid (DOTA), was synthesized to enable Cu-free click cycloaddition and subsequent radiometric isotopic dilution to quantify the number of azide moieties on the dif HA backbone (Scheme 2). Briefly, DBCO-amine was reacted with DOTA-NHS (Macrocyclics, Dallas, Tex.) to prepare DOTA-DBCO. The product was purified by high performance liquid chromatography (Hitachi, Japan) and characterized by mass spectrometry. Next, a 5-fold molar excess of DOTA-DBCO was reacted with the azide-functionalized HA via click chemistry in a water/DMSO (1:1) mixture at 37° C. for 6 hours, and then continuously stirred for 2 days at room temperature. The reaction was purified by size-exclusion chromatography with a Zeba spin column (MWCO=7K) to remove unreacted DOTA-DBCO. DOTA-dif HA was then mixed with a carrier-added solution of ⁶⁴CUCl₂ (Washington University School of Medicine, St. Louis, Mo.), as previously described (Hall, et al., 2012; Ghosh, et al., 2013; Ghosh, et al., 2015). Briefly, DOTA-dif HA was added to a solution containing a 10-fold molar excess of non-radioactive CuCl₂ spiked with ⁶⁴Cu. After heating at 50° C. for 1 h, the reaction mixture was analysed by radio-thin-layer chromatography with Whatman chromatography paper (Sigma-Aldrich, St. Louis, Mo.) and a mobile phase of 0.1 M ammonium acetate/0.05 M EDTA.

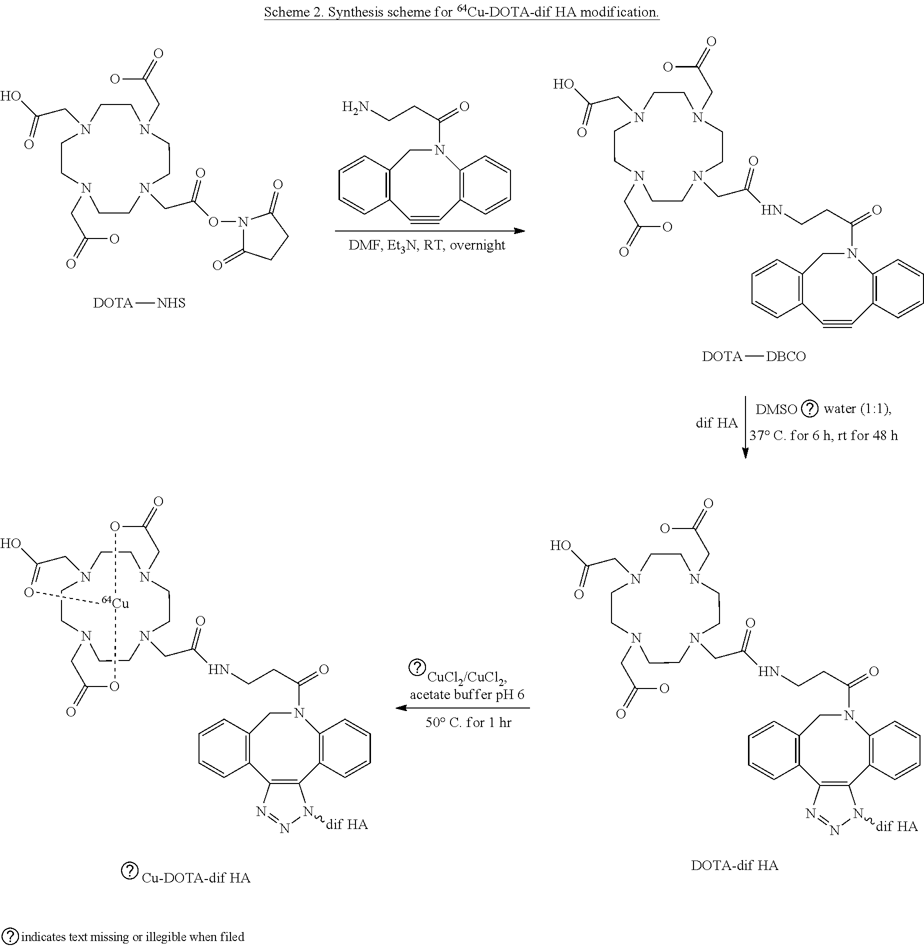

In Vitro Hydrogel Fabrication

Solutions of various ratios of dif HA and mHA were prepared. The total amount of HA in each solution was a constant 1.0% (w/v) in DDW. Hydrogels were formed via photopolymerization by adding 0.1% (w/v) Irgacure 2959 (Ciba Specialty Chemicals, Basel, Switzerland) to the solutions with exposure to 2.3 mJcm⁻² UVA lights for 5 mins.

Hydrogel Characterization

Hydrogel disks formed without cells were evaluated for swelling ratio, water content, and mechanical properties as previously described (Yang, et al., 2015). Briefly, the original mass of the hydrogel disks was measured immediately after fabrication and wet mass of the disks was measured after overnight incubation in neural differentiation media at 37° C., 5% CO₂. The dry mass of the disks was measured after completely drying in a lyophilizer. The swelling ratio was calculated by normalization of the wet mass to the dry mass, while the water content was the ratio of the water mass to the wet mass (Cruise, et al., 1998; Canal and Peppas, 1989).

Young's modulus of the hydrogel disks were quantified using a TA.XTplus Texture Analyzer (Stable Micro Systems, Surrey, UK) with a ¼″ spherical probe, which compressed the samples at a rate of 0.01 mms⁻¹ until 10% strain was reached. Young's modulus (E) was calculated using the following equations (Lin, et al., 2009; Edwin, et al., 2012):

a=R^{1/2}δ^{1/2}

σ=F/(πâ2)

E=3π(1−ν̂2)σ/20ε

Force (F), depth of indentation (δ) and strain (ε) data were recorded to calculate the contact radius (a), indentation stress (σ), and Young's modulus (E). R is the radius of the ¼″ spherical probe. ν is the Poisson's ratio (Khabarov and Selyanin, 2014) (0.5).

Hydrogel Degradation

HA disks (1 mm thickness, 9 mm diameter) were prepared in a manner similar to that described above. Samples (n≥3 per experiment group per time point) were degraded in 100 U hyaluronidase (Sigma Aldrich) per mL of PBS at 37° C. on an orbital shaker at 200 rpm. The amount of uronic acid released during degradation was measured using a previous established carbazole reaction technique from the supernatant until complete dissolution of the hydrogel (Burdick, et al., 2005). Briefly, 100 μl of the degradation solution was added to a 0.025M sodium tetraborate.10H₂O in sulfuric acid solution and heated to 100° C. for 10 min. After adding 100 μl of 0.125% carbazole in absolute ethanol and heating to 100° C. for 15 min, the solution absorbance at 530 nm was measured. The amount of uronic acid was determined using solutions of known concentrations of the 75 kDa HA as a standard. The total amount of uronic acid by standard curve released at each time point is reported.

D3 Mouse Embryonic Stem Cells Culture in Three-Dimensional Hydrogel

mES (D3, passage 68) were encapsulated in the HA solutions at a density of 1×10⁶ cells/mL during photopolymerization. The cell-laden hydrogels were cultivated with 500 μL of pluripotent proliferation media (Dulbecco's modified Eagle's media supplemented with 10% FBS, 0.1 mM β-mercaptoethanol, 0.224 μg/mL L-glutamine, 1.33 μg/mL HEPES, and 1000 units/mL human recombinant Leukemia inhibitory factor) for 1 day, and then cultivated with 500 μL of neural differentiation media (80% F-12, 20% neurobasal medium, 1% penicillin/streptomycin, 0.8×N2, 0.2×B27, 10 mM sodium pyruvate and 2 μM retinoic acid) to induce neural differentiation for the duration of the experiments. Pluripotent proliferation media for cell growth testing was changed every day, and neural differentiation media for differentiation experiments was changed every other day. Cell-laden hydrogel disks were harvested at days 3 and 6. mES from the original population before neural differentiation cultured on 0.1% gelatin-coated tissue culture plastic in pluripotent proliferation media was used as a control group

Capacity for Proliferation and Neural Differentiation of mES in 3D HA Hydrogels In Vitro

To assess the cell proliferation in HA hydrogel disks, a MTS luminescence assay was used on day 0, 3, and 6 according to the manufacturer's protocol (Promega, Madison, Wis., USA). The doubling time of mES in hydrogels was calculated as previously described (Lim, et al., 2015). At designated time points, mES containing HA hydrogel disks were fixed with 4% paraformaldehyde for 30 mins, and the cell membranes then were permeabilized using PBS with 1× Tween20 for 1 hr. Nonspecific antibody binding was minimized with incubation in 5% donkey serum in phosphate buffered saline (PBS) for 1 hr. The hydrogel disks were then incubated in purified rabbit anti-mouse neuron-specific class IIIβ-tubulin antibody (Covance, Princeton, N.J., TUJ1, 1:500) in PBS at 4° C. overnight. Fluorescently conjugated donkey anti-rabbit IgG secondary antibody (Life Technologies, Carlsbad, Calif., 1:500) and nuclei stain draq5 (1:1000) were applied for 1 hr. All incubation steps were carried out on an orbital shaker agitated at 60 rpm with several PBS washes between each step. Images were taken using a confocal microscope (Leica, Surrey, Canada), and percentage of TUJ1 positive cells were quantified using a LAS AF Lite program (Leica, Surrey, Canada). A minimum of 400 cells per sample and 3 different samples for each group were used for quantification of TUJ1 positive cells.

RNA was extracted from the homogenized lysate of the cell-laden HA hydrogel using Trizol reagent (Life Technologies, Carlsbad, Calif.) according to the manufacturer's protocol. Following RNA extraction, 200 ng of isolated RNA from each sample was reverse transcribed into cDNA using amfiRivert Platinum One cDNA Synthesis Master Mix (GenDEPOT, Houston, Tex.) according to the manufacturer's protocol. For real-time polymerase chain reaction (PCR), 10 μL of SYBR Green Master Mix (Applied Biosystems, Foster City, Calif.), 2 pM of each forward and reverse primer, and a cDNA template corresponding to 10 ng of total RNA was placed in 96-well plate. Every reaction was run in duplicate to assure technical reproducibility. The primer sequences of the candidate genes and GAPDH (internal control) are shown in Table 7. SYBR Green PCR conditions were 95° C. for 10 min, followed by 40 cycles of 95° C. for 10 sec, 58° C. for 50 sec, and 72° C. for 20 sec. The 2^{-ΔΔCt} method was used for analysis of the relative changes in gene expression.

TABLE 7
Primer sequences for real-time PCR

| Gene | Sequences | SEQ ID NO |
|---|---|---|
| GAPDH | 5′- TGTGTCCGTCGTGGATCTGA - 3′ | 6 |
| | 5′- CCTGCTTCACCACCTTCTTGA - 3′ | 7 |
| PAX6 | 5′- AAATCCGAGACAGATTACTTCGC - 3′ | 8 |
| | 5′- AAGTCCCGGGATACCAACCA - 3′ | 9 |
| TUJ1 | 5′- TGAGGCCTCCTCTCACAAGT - 3′ | 10 |
| | 5′- GTCGGGCCTGAATAGGTGTC - 3′ | 11 |
| MAP2 | 5′- CTCTCTACCTCCGCTTCCCT - 3′ | 12 |
| | 5′- GCAGAGCTAGAGCTTCTCCG - 3′ | 13 |

Implantation of HA Hydrogel in Rat Spinal Cord Injury

All animal care and surgical interventions were undertaken in strict accordance with the Public Health Service Policy on Humane Care and Use of Laboratory Animals, Guide for the Care and Use of Laboratory Animals and with the approval of the Animal Welfare Committee at the University of Texas Health Science Center at Houston. Eighteen female Fisher 344 rats received 150 kdyne injuries (Cao, et al., 2005; Kar, et al., 2016; Royce Hynes, et al., 2007). Rats were anesthetized via intraperitoneal injection of Nembutal (50 mg/kg) and a dorsal laminectomy was performed at the ninth thoracic vertebra level (T9) to expose the spinal cord. The exposed vertebral column was stabilized by clamping the rostral T8 and caudal T10 vertebral bodies with forceps. Before placing the animal under the IH impactor and securing it for contusion injury, the forceps were carefully adjusted to level the spinal cord in the horizontal plane. After the injury, actual injured forces were recorded and there was less than 0.05% variation between these values. After surgery, animals were given 10 cc of sterile saline subcutaneously and 0.1 cc of gentamycin intramuscularly before being placed on an 87Z heating pad until full recovery from anesthesia. Postoperative care included the manual expression of bladders twice a day for 10 days after which the bladder function returned and injections of Gentamycin (0.1 cc) once a day for up to 1 week. Two weeks post injury the rats were anesthetized and the spinal cords re-exposed at T9. A single 10 μL volume of either sterile saline or 1:2 dif HA:mHA was injected into the injured spinal cord at a rate of 0.5 μl/min through a glass micropipette with outer diameter 50-70 um and the tip sharp-beveled to 30-50°. One and four weeks post-implantation of the hydrogel, spinal cords were collected and fixed using 10% neutral-buffered formalin for 24 h and transferred to 30% sucrose solution for an additional 24 h. The spinal cord was placed in Tissue-Tek OCT, frozen in −80° C., cross-sectioned (20 um), and then washed, blocked in 5% BSA for 1 h at room temperature. Antibodies against EDI (Millipore, Billerica, Mass., USA), 1:200), GFAP (Immunostar, Hudson, Wis., USA, 1:100), and pan-axonal neurofilament (Biolegend, San Diego, Calif., USA, 1:200) were applied overnight at 4° C. The next day, sections were washed 3 times with PBS, and Alexa Fluor secondary antibodies were applied at a dilution of 1:2000 for 4 h at room temperature. Next, slides were washed 3 times with PBS and slides were mounted using a mounting solution containing 4′,6-diamidino-2-phenylindole (DAPI, Thermo Fisher) to assess nuclear staining. In order to quantify the staining of markers at the injury site, a series of images for whole spinal cord were captured using 4× objective using an inverted fluorescence microscope (Nikon TE2000-E, Tokyo, Japan), and then merged by Adobe Photoshop. The lesion site was determined by staining for GFAP, and lesion border measured from a series of longitudinal slices taken every 200 m across the spinal cord. The lesion volume was then determined using the Cavalieri method, where the sum of the cross-sectional areas is multiplied by the distance between tissue sections to determine the volume (Cao, et al., 2005; Michel and Cruz-Orive, 1988; Fan, et al., 2012). A previously reported custom Matlab script determined the injury area and positively stained areas above a user defined threshold for ED1, GFAP, and pan-axonal neurofilament from slices taken across the spinal cords every 200 μm (Wilems, et al., 2015; Wilems and Sakiyama-Elbert, 2015; McCreedy, et al., 2014). The exposure time and gain were not changed during imaging for individual stains.

To confirm HA hydrogel persistence in vivo, eosin-Y was conjugated to both difHA and mHA through the following procedure. 1% dif HA and 1% mHA solution in 0.1M sodium bicarbonate buffer (pH 9) were prepared, separately. To each solution, 250 μL of eosin Y (12 mg/mL) in anhydrous dimethyl sulfoxide was added, and reacted overnight at 4° C. with continuously stirring. The reaction was stopped with 263 μL of 1M ammonium chloride solutions and then incubated for 2 hrs at 4° C. The solutions were dialyzed in ultrapure water for 2 days using 500 kDa molecular weight cutoff membrane, and then lyophilized. Eosin-Y labelled 1:2 dif HA:mHA hydrogel precursor solution was injected into spinal cord injuries and then sectioned and stained with additional antibodies after animal sacrifice as described above.

Statistical Analysis Statistical data is presented as mean±standard error of the mean. One- or Two-way ANOVA followed by Tukey's or Bonferroni's multiple comparisons test, respectively, were performed using GraphPad Prism version 5.0 for Mac (GraphPad Software, La Jolla, Calif.). Significance was defined as p<0.05 in all tests.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aiga et al., Journal of Biological Chemistry, 286:851-8, 2011.
Al'Qteishat et al., Neuro Report, 17:1111-4m 2006a.
Ali et al., Acta Biomaterialia, 25, 253-267, 2015.
Al'Qteishat et al., Brain, 129:2158-76, 2006b.
Ambrosio et al., Journal of Macromolecular Science—Pure and Applied Chemistry, 36 A, 991-1000, 1999.
Austin et al., Biomaterials, 33, 4555-4564, 2012.
Baier Leach et al., Biotechnol Bioeng, 82(5): 578-589, 2003.
Balgude et al., Biomaterials, 22, 1077-1084, 2001.
Banerji et al., Nature Structural & Molecular Biology, 14, 234-239, 2007.
Barbosa da Fonseca et al., Migration and homing of bone-marrow mononuclear cells in chronic ischemic stroke after intra-arterial injection 221:122-8, 2010.
Bencherif et al., Biomaterials, 29, 1739-1749, 2008.
Bergman and Loxley, Clinica Chimica Acta, 27(2): 347-349, 1970.
Bottos et al., Cell Mol Life Sci, 68:2655-66, 2011.
Bottos et al., Proceedings of the National Academy of Sciences, 106:20782-7, 2009.
Burdick et al., Biomacromolecules, 6, 386-391, 2005.
Buss et al., Brain, 130:940-53, 2007.
Buttery, Molecular and Cellular Neuroscience 14:199-212, 1999.
Camci-Unal et al., J Tissue Eng Regen Med, 7, 337-347, 2013.
Canal Peppas, Journal of Biomedical Materials Research, 23, 1183-1193, 1989.
Cao et al., Exp Neurol, 191 Suppl 1, S3-S16 2005.
Chan et al., Soft Matter, 8, 1492-1498, 2012.
Chen et al., Protein Expression and Purification, 81:18-24, 2012.
Choh et al., Biomacromolecules, 12, 1126-1136, 2011.
Chung, et al., J Biomedical Materials Research, Part A, 77, 518-525, 2006
Collins and Birkinshaw, Carbohydrate polymers, 92, 1262-1279, 2013.
Colognato et al., Trends in Neurosciences 28:480-6, 2005.
Crescenzi et al., Biomacromolecules 8:1844-50, 2007.
Cruise et al., Biomaterials, 19, 1287-1294, 1998.
Dinsmore and Solomon, Cell, 64, 817-826, 1991.
Dityatev et al., Trends in Neurosciences, 33:503-12, 2010.
Doetsch, Current Opinion in Genetics & Development 13:543-50, 2003.
Du et al., Journal of Biomedical Materials Research Part A n/a-n/a, 2013.
Fairbanks et al., Macromolecules, 43, 4113-4119, 2010.
Egles et al., Molecular and Cellular Neuroscience, 34:288-98, 2007.
Elbert and Hubbell, Biomacromolecules, 2(2): 430-441, 2001.
Fan et al., International Journal of Biological Sciences, 9, 78-93, 2012.
Fischer et al., Developmental Brain Research, 25, 99-109, 1986.
Frazier et al., Open Glycosci 1: 31-39, 2008.
Fujiwara et al., Polymer International 49:1604-8, 2000.
Gabriel et al., J Nucl Med, 48(4): 508-518, 2007.
Gamini et al., Biomaterials, 23:1161-7, 2002.
Ganios et al., 2013.
Garcia-Alonso et al., Development, 122(9): 2611-2621, 1996.
Ghosh et al., Nuclear Medicine and Biology, 42(2): 177-183, 2015.
Ghosh et al., Journal of Medicinal Chemistry, 56(2): 406-416, 2013.
Gjorlund et al., The FASEB Journal, 26:4174-86, 2012.
Group et al., Neurorehabilitation and Neural Repair, 23:97-107, 2009.
Gupta et al., Biomaterials, 27, 2370-2379, 2006.
Hall et al., Prostate, 72, 129-146, 2012.
Hensarling et al., Journal of the American Chemical Society, 131, 14673-14675, 2009.
Holloway et al., Journal of Controlled Release, 191(0): 63-70, 2014.
Hoogenboom, Angew Chem Int Ed Engl, 49, 3415-3417, 2010.
Horn et al., J Neurosurg Spine, 6, 133-140, 2007.
Homer and Gage, Nature, 407:963-70, 2000.
Hou et al., Journal of Neuroscience Methods, 148, 60-70, 2005.
Hynes et al., J Biomater Sci Polym Ed, 18, 1017-1030, 2007.
Ichtchenko et al., Cell, 81:435-43, 1995.
Irie et al., Science, 277:1511-5, 1997.
Jiang et al., Ann Biomed Eng, 36, 1565-1579, 2008.
Jin et al., Molecular and Cellular Neuroscience 24:171-89, 2003.
Jin et al., Journal of Cerebral Blood Flow and Metabolism, 30:534-44, 2010.
Johnson et al., Biomacromolecules 11:3007-13, 2010.
Kar et al., Biomaterials, 77, 186-197, 2016.
Khabarov and Selyanin, Journal, 216, 2014.
Khaing et al., Journal of Neural Engineering, 8, 2011.
Khetan et al., Soft Matter, 5(8): 1601-1606, 2009.
Kochlamazashvili et al., Neuron, 67:116-28, 2010.
Kushchayev et al., Journal of Neurosurgery: Spine, 0, 1-11.
Kushchayev et al., Journal of Neurosurgery: Spine, 25, 114-124, 2016.
Lam et al., Advanced functional materials, 24, 7053-7062, 2014.
Leipzig and Shoichet, Biomaterials, 30, 6867-6878, 2009.
Li et al., Glia 58:1610-9, 2010.
Liang et al., Biomaterials, 34, 5521-5529, 2013.
Liesi et al., Journal of Neuroscience Research 66:1047-53, 2001.
Lim et al., Cell Transplantation, 24, 1887-1900, 2015.
Lin et al., Biomaterials, 32, 9685-9695, 2011.
Lin et al., Biomech Model Mechanobiol, 8, 345-358, 2009.
Lin et al., Cascading Biomacromolecules, 14:2857-65, 2013.
Liu et al., Glia, 40:25-43, 2002.
Liu et al., World Neurosurgery, 87, 124-131, 2016.
Mahmood et al., Neurosurgery 53:697-703, 2003.
Marchand and Woerly, Neuroscience, 36:45-60, 1990.
Margolis et al., Biochemistry, 14:85-8, 1975.
McCreedy et al., Biomater Sci, 2, 1672-1682, 2014.
McKinnon et al., Biomaterials Science, 1, 460-469, 2013.
Meares et al., Analytical Biochemistry, 142, 68-78, 1984.
Meiners and Mercado, Mol Neurobiol 27:177-95, 2003.
Mergy et al., Journal of Polymer Science Part A: Polymer Chemistry, 50, 4019-4028, 2012.
Michel and Cruz-Orive, Journal of Microscopy, 150, 117-136, 1988.
Moore and Sheetz, Developmental Neurobiology, 71, 1090-1101, 2011.
Mothe et al., Biomaterials, 34, 3775-3783, 2013.
Nakagomi et al., European Journal of Neuroscience 29:1842-52, 2009.
Nakayama et al., European Journal of Neuroscience 31:90-8, 2010.
Norman and Aranda-Espinoza, Cel. Mol. Bioeng., 3, 398-414, 2010.
Osanai et al., Neurosurgery 66:1140-7, 2010.
Pan et al., Journal of Neuroscience Research, 87:3207-20, 2009.
Peixoto et al., Neuron, 76:396-409, 2012.
Prestwich et al., Journal of Controlled Release, 53:93-103, 1998.
Rissone et al., A, Thrombosis, and Vascular Biology, 32:1563-72, 2012.
Roger et al., Circulation, 125:188-97, 2012.
Rubinos and Ruland, Current Neurology and Neuroscience Reports, 16, 1-18, 2016.
Samarelli et al., The Journal of Biological Chemistry, 2014.
Schante, et al., Carbohydrate Polymers, 87, 2211-2216, 2012.
Schnell et al., Frontiers in Neuroscience, 8, 2014.
Schnell et al., PLoS ONE, 7, 2012.
Serban et al., Biomaterials 29:1388-99, 2008.
Shi, R., Neurosci Bull, 29:460-6, 2013.
Shichinohe et al., Brain Research Protocols, 13:166-75, 2004.
Shichinohe et al., Brain Research, 1183:138-47, 2007.
Shih and Lin, Biomacromolecules, 13, 2003-2012, 2012.
Shu et al., Journal of Biomedical Materials Research Part A, 68A(2): 365-375, 2004.
Shu et al., Biomacromolecules, 3, 1304-1311, 2002.
Shu et al., Biomaterials, 25, 1339-1348, 2004.
Slaughter et al., Adv Mater, 21:3307-29, 2009.
Smeds and Grinstaff, Journal of Biomedical Materials Research, 54:115-21, 2001.
Smith Callahan et al., Biomaterials, 34:9089-95, 2013d.
Smith Callahan et al., Acta Biomaterialia, 9(4): 6095-6104, 2013a.
Smith Callahan et al., Acta Biomaterialia, 9:7420-8, 2013b.
Smith Callahan et al., Biomaterials Science 1:537-44, 2013c.
Smith Callahan, Neural Regeneration Research, 11, 66-68, 2016.
Smith Callahan, Policastro et al., Biomacromolecules 14:3047-54, 2013d.
Smith et al., Biomaterials 31:5526-35, 2010.
Smith et al., Biomaterials 30:2516-22, 2009.
Spector, Biomedical Materials, 9:010201, 2014.
Sudhof, Nature, 455:903-11, 2008.
Sun et al., Angew Chem Int Ed Engl, 54, 5981-5984, 2015.
Takahashi et al., Biomacromolecules, 14, 3581-3588, 2013.
Tamura et al., J Nucl Med 54(11): 1869-1875, 2013.
Timpl and Brown, BioEssays, 18:123-32, 1996.
Tingaut et al., Journal of Materials Chemistry, 21, 16066-16076, 2011.
Vanderhooft, et al., Macromolecular Bioscience, 9, 20-28, 2009.
Volpato et al., Biomaterials, 34, 4945-4955, 2013.
von Leden et al., Journal of Neurotrauma, DOI: 10.1089/neu.2016.4486, 2016.
Wang et al., Interface focus, 2, 278-291, 2012.
Wilems and Sakiyama-Elbert, J Control Release, 213, 103-111, 2015.
Wilems et al., Acta Biomater, 28, 23-32, 2015.
Witiw and Fehlings, Journal Of Spinal Disorders & Techniques, 28, 202-210, 2015.
Yang et al., Cancer Biother Radiopharm, 19(4): 443-456, 2004.
Yang et al., Acta Biomater, 21, 55-62, 2015.
Yasuda et al., Journal of Neurosurgery, 112:336-44, 2010.
Young et al., Acta Biomaterialia, 9, 7151-7157, 2013.
Zhang et al., Acta Biomaterialia, 30, 188-198, 2016.
Zheng et al., Polymer Chemistry, 4(7): 2215-2218, 2013.
Zheng et al., ACS Macro Letters 1:1071-3, 2012.
Zustiak et al., Acta Biomaterialia 6(9): 3404-3414, 2010.

What is claimed is:

1. A compound of the formula: 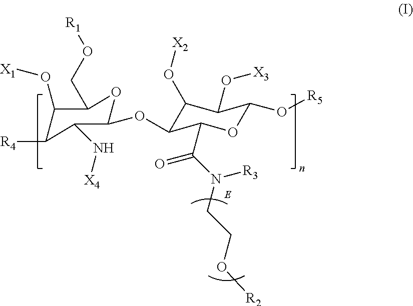
wherein:
R₁ is —Y₁-L₁, wherein:
Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
L₁ is a first linking group;
R₂ is —Y₂-L₂, wherein:
Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
L₂ is a second linking group;
R₃ is hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
X₁, X₂, and X₃ are each independently hydrogen, alkyl_{(C≤6)}, substituted alkyl_{(C≤6)}, or a hydroxy protecting group;
X₄ is hydrogen, acyl_{(C≤6)}, substituted acyl_{(C≤6)}, or a monovalent amino protecting group;
m is 1-20; and
n is 1-5000;
provided that the first linking group and the second linking group are different;
or a pharmaceutically acceptable salt thereof.

2. The compound of claim 1, wherein the compound is further defined as: 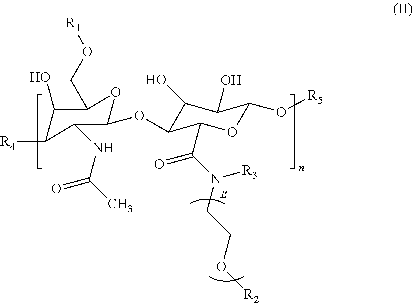
wherein:
R₁ is —Y₁-L₁, wherein:
Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
L₁ is a first linking group;
R₂ is —Y₂-L₂, wherein:
Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
L₂ is a second linking group;
R₃ is hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
m is 1-20; and
n is 1-5000;
provided that the first linking group and the second linking group are different;
or a pharmaceutically acceptable salt thereof.

3. The compound of either claim 1 or claim 2, wherein the compound is further defined as: 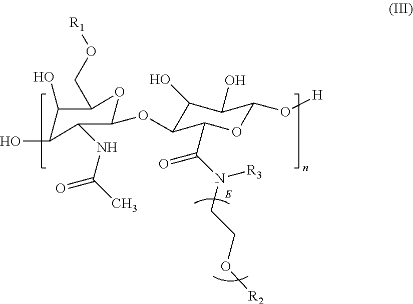
wherein:
R₁ is —Y₁-L₁, wherein:
Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
L₁ is a first linking group;
R₂ is —Y₂-L₂, wherein:
Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
L₂ is a second linking group;
R₃ is hydrogen, alkyl_{(C≤6)}, or substituted alkyl_{(C≤6)};
m is 1-20; and
n is 1-5000;
provided that the first linking group and the second linking group are different;
or a pharmaceutically acceptable salt thereof.

4. The compound according to any one of claims 1-3, wherein Y₁ is alkanediyl_{(C≤12)} or substituted alkanediyl_{(C≤12)}.

5. The compound of claim 4, wherein Y₁ is —CH₂CH₂—.

6. The compound according to any one of claims 1-5, wherein L₁ is —SH.

7. The compound according to any one of claims 1-5, wherein L₁ is an acrylate or a methylacrylate group.

8. The compound of claim 7, wherein L₁ is:

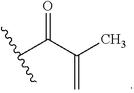 [SMILES: *C(=O)C(=C)C]

9. The compound according to any one of claims 1-8, wherein Y₂ is alkanediyl_{(C≤12)} or substituted alkanediyl_{(C≤12)}.

10. The compound of claim 4, wherein Y₂ is —CH₂CH₂—.

11. The compound according to any one of claims 1-11, wherein L₂ is —N₃.

12. The compound according to any one of claims 1-11, wherein R₃ is hydrogen.

13. The compound according to any one of claims 1-12, wherein m is 2, 3, or 4.

14. The compound according to any one of claims 1-13, wherein n is an integer between 1 and 1000.

15. The compound of claim 14, wherein n is an integer between 1 and 500.

16. The compound according to any one of claims 1-13, wherein the compound is further defined as: 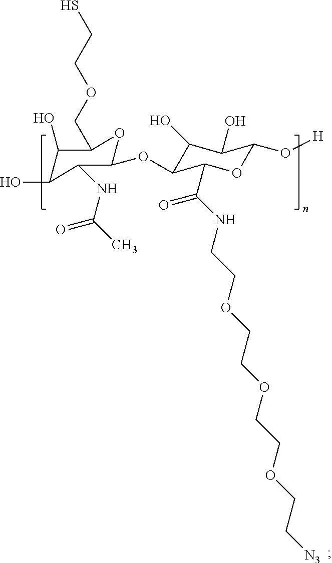
or a pharmaceutically acceptable salt thereof.

17. A hydrogel comprising a compound in accordance with claim 1.

18. The hydrogel of claim 17, comprising two hyaluronic units comprising:
a first hyaluronic unit of the formula: 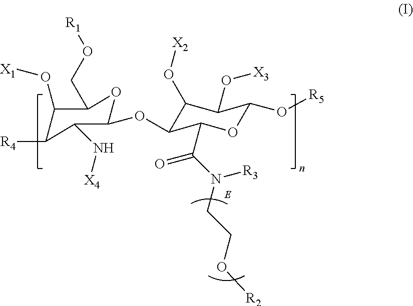
wherein:
R₁ is —Y₁-L₁, wherein:
Y₁ is a covalent bond, alkanediyl_{(C≤12)}, alkenediyl_{(C≤12)}, alkynediyl_{(C≤12)}, arenediyl_{(C≤12)}, or a substituted version of any of these groups; and
L₁ is a first linking group;
R₂ is —Y₂-L₂, wherein:
Y₂ is a covalent bond, alkanediyl_{(C≤12)}, alkenediyl_{(C≤12)}, alkynediyl_{(C≤12)}, arenediyl_{(C≤12)}, or a substituted version of any of these groups; and
L₂ is a second linking group;
R₃ is hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
X₁, X₂, and X₃ are each independently hydrogen, alkyl_{(C≤6)}, substituted alkyl_{(C≤6)}, or a hydroxy protecting group;
X₄ is hydrogen, acyl_{(C≤6)}, substituted acyl_{(C≤6)}, or a monovalent amino protecting group;
m is 1-20; and
n is 1-5000; and
a second hyaluronic unit of the formula: 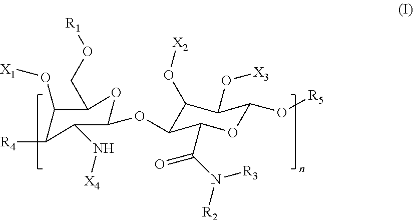
wherein:
R₁ is —Y₃-L₃, wherein:
Y₃ is a covalent bond, alkanediyl_{(C≤12)}, alkenediyl_{(C≤12)}, alkynediyl_{(C≤12)}, arenediyl_{(C≤12)}, or a substituted version of any of these groups; and
L₃ is a third linking group;
R₂ and R₃ are each independently hydrogen, alkyl_{(C≤6)}, or substituted alkyl_{(C≤6)};
R₄ is hydroxy, alkoxy_{(C≤6)}, or substituted alkoxy_{(C≤6)};
R₅ is hydrogen, alkyl_{(C≤6)}, or substituted alkyl_{(C≤6)};
X₁, X₂, and X₃ are each independently hydrogen, alkyl_{(C≤6)}, substituted alkyl_{(C≤6)}, or a hydroxy protecting group;
X₄ is hydrogen, acyl_{(C≤6)}, substituted acyl_{(C≤6)}, or a monovalent amino protecting group;
m is 1-20; and
n is 1-5000;
provided that the first, second, and third linking groups are all different; and
wherein the hydrogel comprises a ratio of the first hyaluronic acid to the second hyaluronic acid from about 10:1 to about 1:10.

19. The hydrogel of claim 18, wherein the first linking group comprises a thiol unit.

20. The hydrogel of either claim 18 or claim 19, wherein the second linking group comprises an azide group.

21. The hydrogel according to any one of claims 18-20, wherein the third linking group is a methacrylate.

22. The hydrogel according to any one of claims 18-21, wherein the ratio is from about 1:1 to about 1:7.5.

23. The hydrogel of claim 22, wherein the ratio is from about 1:2 to about 1:5.

24. The hydrogel according to any one of claims 18-23, wherein the first linking group, the second linking group, or the third linking group is linked to a polypeptide.

25. The hydrogel of claim 24, wherein the polypeptide is derived from laminin, neuroligin-1, fibronectin, collagen, n-cadherin, cartilage oligomeric protein, neural cell adhesion molecule 1, victronectin, brain-derived neurotrophic factor, neurexin, insulin-like growth factor, link binding protein, bone morphgenic proteins, decoriin, or aggrecan binding peptides.

26. The hydrogel of claim 25, wherein the polypeptide comprises the sequence IKVAV (SEQ ID NO:1.

27. The hydrogel according to any one of claims 18-23, wherein the first linking group, the second linking group, or the third linking group is linked to an imaging agent.

28. The hydrogel of claim 27, wherein the imaging agent is a chelating group bound to a radioisotope.

29. The hydrogel of claim 27, wherein the imaging agent is fluorophore.

30. A composition comprising:
(a) a hydrogel according to any one of claims 18-29; and
(b) a cell.

31. The composition of claim 30, wherein the cell is a stem cell.

32. The composition of claim 31, wherein the stem cell is a human induced pluripotent stem cell.

33. The composition of claim 30, wherein the cell is a neural cell or a neural stem cell.

34. The composition according to any one of claims 30-33, wherein the cell is encapsulated in the hydrogel.

35. A method of treating a disease or disorder in a patient comprising implant a hydrogel or composition according to any one of claims 18-34 into a tissue of the patient.

36. The method of claim 35, wherein the disease or disorder is a neurological disease or disorder and wherein the hydrogel or composition is implanted in a neuronal tissue.

37. The method of claim 36, wherein the neurological disease or disorder is a brain injury.

38. The method of claim 36, wherein the neurological disease or disorder is a stroke.

39. The method of claim 36, wherein the neurological disease or disorder results in a neuronal lesion.

40. A method of promoting cell growth in a tissue lesion comprising implanting a hydrogel or composition according to any one of claims 18-34 into the tissue lesion under conditions sufficient to promote cell or tissue growth.

41. The method of claim 40, further defined as a method of promoting neuronal growth in a neuronal lesion comprising implanting the hydrogel or composition into the neuronal lesion under conditions sufficient to promote neuronal tissue formation.

42. The method of claim 40, wherein the method comprises implanting a composition.

43. A method of inducing differentiation in a stem cell comprising contacting the stem cell with a hydrogel according to any one of claims 18-29 under conditions sufficient to cause the stem cell to differentiate into a mature cell.

44. The method of claim 43, wherein the stem cell is a neuronal stem cell.

45. The method of claim 44, comprising encapsulating the neuronal stem cell with the hydrogel.

46. The method of claim 44, wherein the mature cell is a mature neuron, oligodendrocyte, or astrocyte.

47. The method according to any one of claims 35-46, wherein the method is performed in vitro.

48. The method according to any one of claims 35-46, wherein the method is performed in vivo.

49. The method according to any one of claims 35-48, wherein the method is performed in a patient.

50. The method of claim 49, wherein the patient is a mammal.

51. The method of claim 50, wherein the patient is a human.

52. The method according to any one of claims 35-39 and 43-51, wherein the method comprises implanting the hydrogel or the composition according to any one of claims 18-34 into a neuron or neuronal tissue.


SEQUENCE LISTING (15 sequences)

SEQ ID NO: 1 - protein, 5 aa, Artificial sequence
  Synthetic peptide
IKVAV

SEQ ID NO: 2 - protein, 5 aa, Artificial sequence
  Synthetic peptide
YIGSR

SEQ ID NO: 3 - protein, 10 aa, Artificial sequence
  Synthetic peptide
RNIAEIIKDI

SEQ ID NO: 4 - protein, 17 aa, Artificial sequence
  Synthetic peptide
SEGNRWSNSTKGLFQRA

SEQ ID NO: 5 - protein, 3 aa, Artificial sequence
  Synthetic peptide
LRE

SEQ ID NO: 6 - DNA, 20 bp, Artificial sequence
  Synthetic primer
tgtgtccgtcgtggatctga

SEQ ID NO: 7 - DNA, 21 bp, Artificial sequence
  Synthetic primer
cctgcttcaccaccttcttga

SEQ ID NO: 8 - DNA, 23 bp, Artificial sequence
  Synthetic primer
aaatccgagacagattacttcgc

SEQ ID NO: 9 - DNA, 20 bp, Artificial sequence
  Synthetic primer
aagtcccgggataccaacca

SEQ ID NO: 10 - DNA, 20 bp, Artificial sequence
  Synthetic primer
tgaggcctcctctcacaagt

SEQ ID NO: 11 - DNA, 20 bp, Artificial sequence
  Synthetic primer
gtcgggcctgaataggtgtc

SEQ ID NO: 12 - DNA, 20 bp, Artificial sequence
  Synthetic primer
ctctctacctccgcttccct

SEQ ID NO: 13 - DNA, 20 bp, Artificial sequence
  Synthetic primer
gcagagctagagcttctccg

SEQ ID NO: 14 - protein, 6 aa, Artificial sequence
  Synthetic peptide
GIKVAV

SEQ ID NO: 15 - protein, 7 aa, Artificial sequence
  Synthetic peptide
CGGGERL